(19) United States
(12) Patent Application Publication
(10) Pub. No.: US 2019/0298688 A1
(43) Pub. Date: Oct. 3, 2019

(54) PUTRESCINE SLOW-RELEASE TOPICAL FORMULATIONS

(71) Applicant: VIVIER CANADA INC., VAUDREUIL-DORION (CA)

(72) Inventor: GHISLAIN VIVIER, ST-LAZARE (CA)

(21) Appl. No.: 16/335,109

(22) PCT Filed: Nov. 21, 2017
(86) PCT No.: PCT/CA2017/051388

§ 371 Date: Mar. 20, 2019

Related U.S. Application Data
(60) Provisional application No. 62/424,681, filed on Nov. 21, 2016.

(51) Int. Cl. A61K 31/375; A61K 36/752; A61K 47/10; A61K 47/26; A61K 47/18; A61K 31/132; A61P 17/02; A61Q 19/00; A61Q 19/08; A61K 8/67; A61K 8/41; A61K 8/34; A61K 8/60; A61K 8/9789

(52) U.S. Cl. CPC A61K 31/375; A61K 8/9789; A61K 47/10; A61K 47/26; A61K 47/186; A61K 31/132; A61P 17/02; A61Q 19/008; A61Q 19/007; A61Q 19/08; A61K 8/676; A61K 8/41; A61K 8/345; A61K 8/416; A61K 8/60; A61K 36/752

(57) ABSTRACT

The present invention describes slow-release compositions for the effective delivery of active ingredients such as putrescine and Vitamin C into the skin. These compositions may be used in a variety of cosmetic and therapeutic applications including for promoting wound healing, for reducing or preventing the formation of hypertrophic scar tissue, for reducing or preventing skin irritation and inflammation and/or for reducing or preventing skin's signs of aging.

### CROSS REFERENCE TO RELATED APPLICATIONS

This application claims priority of U.S. provisional application Ser. No. 62/424,681, filed on Nov. 21, 2016, which is incorporated herein by reference in its entirety.

### FIELD OF THE INVENTION

The present invention relates to stable, slow-release formulations comprising primary polyamines and/or Vitamin C and uses thereof for stimulating wound healing, thickening skin and reducing signs of aging such as fine lines and wrinkles. The present invention also relates to a process for obtaining such formulations.

### BACKGROUND OF THE INVENTION

Skin is a physical barrier to the environment. In mammals, it is composed of multiple layers of ectodermal tissue, and guards the underlying muscles, bones, ligaments and internal organs. It is the alteration of the barrier properties and actual damage to this barrier that causes skin conditions.

The epidermis and the dermis, separated by the basal membrane, EDJ (epidermal-dermal junction) or Grenz Zone, constitute the cutaneous covering on the hypoderm. The epidermis is the most superficial layer of the skin and provides its resistance and impermeability. Alteration of this layer will negatively affect perceived quality of the skin and will eventually lead to cutaneous aging. (the sun, lazers, retinoids, chemical peels, microdermabrasion, skin exfoliants (glycolic, salicylic acid, AHAs/BHAs) causes thinning) The dermis, the internal layer of the skin, is conjunctive tissue composed of cells (essentially fibroblasts) dispersed in a complex medium called the extracellular matrix (ECM). This matrix consists of collagen and elastin fibers, glycoproteins (fibronectin and laminin) and proteoglycans. The extracellular matrix serves as a structure for the cells, allowing tissues and organs to cohere in pluricellular organisms. The EDJ, which attaches the epidermis and the dermis of the skin is vitally important due to the roles it plays in cellular communication, nutrient exchange and absorption and other skin functions. The layers of the epidermis are continually moving upward, throwing their “contents” overboard, flattening, building up at the surface and then eventually sloughing off to make room for the cells right behind them. This natural movement or “keratinization” of the skin is an integral part of skin renewal and healing. It would not be possible without the epidermal-dermal Junction (EDJ) maintaining the relationship between the two main layers of skin, allowing for healthy communication from the top all the way to the bottom.

The EDJ is also responsible for the exchange of nutrients back and forth from the epidermis to the dermis. These nutrients are carried in the blood from the food we eat and absorbed through the pores from topical application. Vitamins, antioxidants, acids and other nutrients are needed for DNA repair, new cell production, protection from outside elements and oxidative stress and more. In youth, this junction is a healthy, wavy terrain. The finger-like waves in the junction, called rete ridges, form the interlocking connection between the dermis and epidermis. They increase the surface area of the epidermis that is exposed to these blood vessels and the needed nutrients. Without this nutrient exchange, skin would suffer premature aging and damage.

As we age or stress our skin, it tends to thinner and to flatten out. If the junction completely flat lines, no pun intended, the communication and nutrient exchange comes to a halt. So, in order to maintain skin healthy—and youth—you want to keep the communication open and the EDJ's rete waves as wavy as possible. Maintaining EDJ functioning can be helped by proper diet and topical skin supplementation as well as limiting over exfoliation, over exposure to harsh elements and any other form of stress or trauma.

Dry skin is one of the most common skin problems which can be treated by applying moisturizers. Moisturizers are oily substances which are used to replace natural skin oils, to cover tiny fissures and to provide a protective film. Four types of moisturizers have been described according to their mechanism of action: Occlusive, Humectants, Emollients, and Protein Rejuvenators. Occlusive moisturizers (e.g., petroleum in a minimum concentration of 5%, lanolin, mineral oil, silicones (such as dimethicone)) are substances which physically block trans-epidermal water loss (TEWL) in the stratum corneum. Humectants (e.g., glycerin, sorbitol, urea, alpha-hydroxy acids, and other sugars) work by attracting trans-epidermal water to the skin to improve hydration of the stratum corneum. However, their chronic use may contribute to continuous evaporation from the skin and may under certain conditions, exacerbate dryness. Emollients (e.g., Vitamin E, fatty acids, cholesterol, squalene, structural lipids (e.g., ceramide), stearic, linoleic, linolenic and lauric acids (found in palm oil and coconut oil) smooth skin by filling spaces between skin flakes and droplets of oil. Some emollients (long chain fatty acids) act by influencing skin's physiology and pathology through their action on skin barrier function, eicosanoid production, cell signaling and membrane fluidity. Moisturizers containing collagen and other large proteins (e.g., elastin and keratin) are said to rejuvenate the skin by providing essential proteins (however, efficient dermal delivery of such proteins often remains a challenge due to their large size). Moisturizers and their effects are reviewed in C. W. Lynde. Moisturizers: What they are and how they work. Skin Therapy Letter, 2001; www.skintherapyletter.com/2001/6.13/2.html.

Cutaneous aging is a complex phenomenon responsible for progressive changes of the skin. Aging of the skin results from two processes: (1) an intrinsic process, corresponding to chronological aging, and (2) an extrinsic process resulting mainly from the deleterious effect of exposure to environmental stresses. Genetic, UV exposure (e.g., sun exposure), climatic factors (harshness/wind/cold/warm), pollution (chemical, free radicals, contaminant, nitrogen oxide, metals), alcohol consumption and smoking are factors involved in cutaneous aging.

Scar tissue is formed during healing of wounds following for example traumatic injury, burn and surgery (including cosmetic surgery). Often unpredictably, hypertrophy of the scar tissue occurs. Hypertrophic scar formation is characterized by the accumulation of collagen type III out of proportion to collagen type I. During skin wound healing it appears that type III procollagen amino peptide (PIIP) is cross-linked to other components of the wound matrix, such as fibrin and fibronectin, by tissue transglutaminase. Such cross-linking is thought to contribute to tissue hypertrophy and disproportionate scarring. Common treatment of hypertrophic scar tissue includes the use of drugs with potentially serious side effects (e.g., corticosteroid injection) and invasive procedures including surgical excision or cryotherapy.

Human growth factors (HGFs) have been attributed to many rejuvenating properties and are used as anti-aging agents and alternative wound healers. Many growth factors such as EGF and TGF-B are large proteins, which do not penetrate the skin well. They are also very unstable and often lose their activity within days in water or even as solids at normal temperatures. Furthermore, there are more and more concerns that at certain concentrations and over certain durations of application they can cause cells to over-proliferate and increase the risk of developing cancer and other health problems.

Primary polyamines (polyazaalkanes) have long been known as antioxidants. Recently, these compounds are attracting more and more interests as they have been shown to reduce skin inflammation and irritation and to be highly effective wound healing agents. Their effect on wound healing and hypertrophic scarring is thought to be due, at least partly, to their transglutaminase inhibiting activity which reduces type III pro-collagen cross-linking to components of the wound extracellular matrix. In addition to their effects on skin irritation, inflammation and on wound healing, primary polyamines have also been identified as useful agents to prevent and/or reduce the skin's signs of ageing (see for example U.S. Pat. No. 5,885,982, CA 2 606 630 and WO 2009/067799).

Examples of primary polyamines include aminoacetonitrile, dansylcadaverine (1,5 diaminopentane), spermidine, and putrescine (1,4 diaminobutane). Putrescine is a natural compound that is related to cadaverine; both are produced by the breakdown of amino acids in living and dead organisms. The two compounds are largely responsible for the foul odor of putrefying flesh but are also found in other conditions (e.g., bad breadth). They are also found in semen and some microalgae, together with related molecules like spermine and spermidine. Putrescine is synthesized in small quantities by healthy living cells by the action of ornithine decarboxylase.

U.S. Pat. No. 5,885,982 (Dolynchuk) describes a method of preventing hypertrophic scar in human dermal wounds by applying a topical inhibitor of fibroblast tissue transglutaminase. Putrescine was shown to reduce collagen cross-linking in vitro and in vivo resulting in a softer and a more rapidly mature-looking scar as compared to controls. The negative side effects, typical of steroid injection, were not seen. Studies done on human harvested scars revealed an increase in apoptosis of scar fibroblasts which lead to a less active scar than seen with other methods of treatment.

Canadian patent application CA 2,606,630 (Dolynchuk, K.) further shows that putrescine provides beneficial effects on the epidermis of eroded skin increasing its barrier function as well as the thickness of the stratum lucidum in animals and the inner strata of human epidermis. The presence of topical polyamines such as putrescine enhances the cellular regenerative mechanisms and creates a robust Genz layer. These are typically reduced by inflammation, steroids and ageing effects, the recovery of which, results in a more youthful looking and functionally stable skin. Vitamin C (also known as ascorbic acid) is another well-known powerful antiaging and wound healing agent. Vitamin C deficiency causes spontaneous breakdown of wounds in the absence of infection in many surgery patients. Furthermore, evidence from the scientific literature shows that Vitamin C can increase collagen production in skin fibroblasts¹, counter skin damage associated with photo aging² and reduce the inflammation and erythema of sunburn³. Ultimately Vitamin C helps reduce the expression of skin aging, translated into the appearance of fine lines or wrinkles in the skin.

In mammals, Vitamin C is involved in all phases of wound healing. It is necessary for a normal response to physiological stressors, with this need increasing during times of injury. Events that cause wounding, including trauma or surgery are physiological stressors that have been associated with a decrease in blood plasma Vitamin C levels. In the inflammatory phase it is required for neutrophil apoptosis and clearance. During the proliferative phase, Vitamin C has been shown to regulate synthesis, maturation, secretion and degradation of collagen. Also, evidence suggests that Vitamin C may improve wound healing by stimulating quiescent fibroblasts to divide and by promoting their migration into the wounded area. Furthermore, studies have shown that Vitamin C protects the skin by increasing the capacity of fibroblasts to repair potentially mutagenic DNA lesions and acts as a powerful antioxidant and immune system modulator.

The numerous beneficial effects attributed to Vitamin C makes it a particularly remarkable active agent in cosmetic and wound healing applications. Humans lack the ability to store Vitamin C, so it is important to continually replenish this vitamin through dietary means and/or other means such as topical supplementation (MacKay, Douglas, N D, and Miller, Alan L., ND, 2003).

Although a variety of chemical forms of Vitamin C are available commercially, not all forms are equally absorbed or active. As an antioxidant, Vitamin C needs to remain in its unoxidized form in order to be effective. However, it is particularly subject to oxidative degradation. Vitamin C is a moderately strong reducing agent, which makes it unstable in aqueous solutions, especially at high pHs. Paradoxically, water is one of the best solvents to dissolve many active ingredients including Vitamin C. Vitamin C is much less soluble in glycols such as propylene glycol (50 mg/ml) and in alcohols such as ethanol (10 mg/ml in absolute ethanol). Although water is the best solvent to provide a Vitamin C solution, it is paradoxically one of the worst to protect it against oxidative damages. The problem to be solved with ascorbic acid formulations has thus always been to find a compromise between solubilization and stability. Furthermore, because of its sensitivity, it can be a challenge to combine Vitamin C (especially in high concentrations) with certain active ingredients, while maintaining adequate stability activity of all components in the formulation.

The creation of stable topical skin care compositions thus often presents many difficulties and challenges due to the nature of the active ingredients and unpredictable interactions between components in the final formulation. Another important challenge in the field of topical formulations (cosmetic and therapeutic) remains the ability to deliver active ingredients into the skin to reach target cells and provide biological effects. Factors that influence skin penetration of a given active ingredient are numerous and include size of the molecule, its lipophilic/hydrophilic nature, polarity, interactions with other components in the composition and skin condition.

Despite the number of solutions that have been proposed, there thus remains a need for novel skin care compositions and methods of use thereof.

### SUMMARY OF THE INVENTION

Accordingly, the present invention provides new skin care compositions which are stable and allow for the efficient penetration and delivery of active ingredients into the skin. These formulations may be used in therapeutic and/or cosmetic applications and are particularly useful in preventing and reducing skin's signs of aging (fine lines and wrinkle), skin irritation and inflammation, promoting skin thickening, promoting wound healing and/or reducing the development of scar tissue, including hypertrophic scar tissue.

More specifically, compositions of the present invention contain Vitamin-C (e.g., L-Ascorbic Acid USP) and 1,4 Diaminobutane (e.g., Polyamine-DAB™) which encourages the natural regenerating process, accelerate healing, promote new cell growth, increase healthy blood flow, and boost collagen and moisture levels in the skin which result in skin thickening and fine lines and wrinkles reduction.

In an aspect, compositions of the present invention focus on reducing inflammation, rebalancing metabolic functions, resulting in a healthier, thicker and younger looking skin. This is achieved through compositions of the present invention comprising Vitamin-C (e.g., L-Ascorbic Acid USP) and polyamines (e.g., 1,4 Diaminobutane. Polyamine-DAB™) along with its efficient IDS or IntraDermal Delivery System. In embodiments, compositions of the present invention contribute to strengthen the immune system; increase skin circulation, improve scar remodeling, repair DNA, replenish natural growth factors, re-establish the protective barrier, restore antioxidant levels, activate collagen at the source, and promote skin thickening resulting in improved skin texture, improved skin tone, improved skin moisture level and the prevention and reduction of fine lines and wrinkles for a healthier and younger looking skin.

In a first aspect, the present invention thus provides a slow-release aqueous topical composition comprising (i) at least one active ingredient, (ii) ethoxydiglycol; (iii) propylene glycol; and (iv) (a) betaine and/or (b) polysorbate 60 or polysorbate 20 (preferably polysorbate 20). In embodiments, at least one active ingredient comprises a primary polyamine (e.g., putrescine) and/or Vitamin C.

In a further aspect, the present invention concerns a slow-release aqueous topical composition comprising (i) at least 16% of Vitamin C; (ii) ethoxydiglycol; and (iii) propylene glycol. In embodiments, the composition further comprises (iv) (a) betaine and/or (b) polysorbate 60 or polysorbate 20 (preferably polysorbate 20).

In embodiments, compositions of the present invention comprise between about 0.5 w/w and about 3% w/w of betaine. In particular embodiments, about 1.2% w/w of betaine. In embodiments, the compositions comprise between about 0.1% w/w and about 3% w/w of polysorbate 60 or polysorbate 20 (preferably polysorbate 20). In particular embodiments, the compositions comprise about 1.3% w/w of polysorbate 60. In particular embodiments, the compositions comprise about 1.3% w/w of polysorbate 20.

In embodiments, the above-noted primary polyamine is a primary aliphatic lower-alkyl (C1-5) monoamine; a primary aliphatic alkylamine or a primary aliphatic lower-alkyl (C1-5) polyamine. In embodiments, the primary aliphatic lower-alkyl (C1-5) monoamine is aminoacetonitrile. In embodiments the primary aliphatic alkylamine is spermine or spermidine. In embodiments, the primary aliphatic lower-alkyl (C1-5) polyamine is putrescine or dansylcadaverine. In preferred embodiments, the primary aliphatic lower-alkyl (C1-5) polyamine is putrescine.

In embodiments, compositions of the present invention comprise between about 0.1% w/w to about 1% w/w of putrescine. In embodiments, about 0.4% w/w of putrescine. In other embodiments, about 0.8% w/w of putrescine.

In embodiments, the above-noted Vitamin C comprises L-ascorbic acid, 3-O-ethyl ascorbic acid (ETVC) or a combination thereof. In particular embodiments, compositions of the present invention comprise more than zero and up to about 20% w/w of 3-O-ethyl ascorbic acid, L-ascorbic acid or a combination thereof as Vitamin C. In embodiments, about 10% w/w of L-ascorbic acid and/or 3-O-ethyl ascorbic acid (ETVC).

In embodiments, compositions of the present invention comprise between 22% and 60% w/w of one or more glycols. The presence of one or more specific glycols is believed to promote delivery of active ingredients (e.g., putrescine and vitamin C (e.g., L-ascorbic acid and/or ETVC) in a stable and suitable form into the skin. In embodiments, compositions of the present invention comprise between 22% and 60% w/w of (i) ethoxydiglycol, (ii) propylene glycol, (iii) butylene glycol, (iv), pentylene glycol, or (v) a combination of at least two of (i) to (iv).

In embodiments, compositions of the present invention comprise up to 2.5% w/w of ethoxydiglycol. In embodiments, compositions of the present invention comprise at least 2.5% w/w of ethoxydiglycol. In embodiments, compositions of the present invention comprise between about 1% w/w and about 2.5% w/w of ethoxydiglycol. In embodiments, compositions of the present invention comprise between about 2.5% w/w and about 48% w/w of ethoxydiglycol. In particular embodiments between about 48% w/w and about 55% w/w of ethoxydiglycol. In embodiments, compositions of the present invention comprise between about 22% w/w and about 60% w/w of ethoxydiglycol. In particular embodiments between about 48% w/w and about 55% w/w of ethoxydiglycol.

Generally, when compositions of the present invention comprise up to 2.5% ethoxydiglycol; propylene glycol, butylene glycol and/or pentylene glycol are included in the compositions at a concentration between about 60% w/w and about 80% w/w. Generally, it is thought that propylene glycol, butylene glycol and pentylene glycol are interchangeable i.e., one can be substituted with the other(s) in compositions of the present invention. Thus, compositions of the present invention may comprise up to about 80% w/w of glycol (i.e., propylene glycol, butylene glycol, pentylene glycol and/or ethoxydiglycol, with preferably at least 2.5% ethoxydiglycol).

Accordingly, compositions of the present invention may comprise between about 0% w/w and about 80% w/w of propylene glycol. In embodiments, compositions of the present invention comprise between about 20% w/w and about 80% w/w of propylene glycol. In embodiments, compositions of the present invention comprise between about 0% w/w and about 22% w/w of propylene glycol. In particular embodiments, between about 0% w/w and about 27.5% w/w of propylene glycol. In embodiments, compositions of the present invention comprise between about 0% w/w and about 44% w/w of propylene glycol. In embodiments, compositions of the present invention comprise between about 0% w/w and about 32% w/w of propylene glycol. In embodiments, compositions of the present invention comprise between about 20% w/w and about 32% w/w of propylene glycol. In embodiments, compositions of the present invention comprise between about 22% w/w and about 32% w/w of propylene glycol. In embodiments, compositions of the present invention comprise between about 22% w/w and about 54% w/w of propylene glycol. In embodiments, compositions of the present invention comprise between about 22% w/w and about 76% w/w of propylene glycol. In embodiments, compositions of the present invention comprise between about 20% w/w and about 30% w/w of propylene glycol. In particular embodiments, between about 26% w/w and about 27.5% w/w of propylene glycol. In particular embodiments, compositions of the present invention comprise about 32% w/w of propylene glycol. In other particular embodiments, compositions of the present invention comprise about 23% w/w of propylene glycol. In other particular embodiments, compositions of the present invention comprise about 44% w/w of propylene glycol. In other particular embodiments, compositions of the present invention comprise about 76% w/w of propylene glycol. In other particular embodiments, compositions of the present invention comprise about 27% w/w of propylene glycol.

Similarly, compositions of the present invention may comprise between about 0% w/w and about 80% w/w of butylene glycol. In embodiments, compositions of the present invention comprise between about 20% w/w and about 80% w/w of butylene glycol. In embodiments, compositions of the present invention comprise between about 0% w/w and about 22% w/w of butylene glycol. In particular embodiments, between about 0% w/w and about 27.5% w/w of butylene glycol. In embodiments, compositions of the present invention comprise between about 0% w/w and about 44% w/w of butylene glycol. In embodiments, compositions of the present invention comprise between about 0% w/w and about 32% w/w of butylene glycol. In embodiments, compositions of the present invention comprise between about 20% w/w and about 32% w/w of butylene glycol. In embodiments, compositions of the present invention comprise between about 22% w/w and about 32% w/w of butylene glycol. In embodiments, compositions of the present invention comprise between about 22% w/w and about 54% w/w of butylene glycol. In embodiments, compositions of the present invention comprise between about 22% w/w and about 76% w/w of butylene glycol. In embodiments, compositions of the present invention comprise between about 20% w/w and about 30% w/w of butylene glycol. In particular embodiments, compositions of the present invention comprise about 22% w/w of butylene glycol. In other particular embodiments, compositions of the present invention comprise about 44% w/w of butylene glycol. In other particular embodiments, compositions of the present invention comprise about 76% w/w of butylene glycol. In other particular embodiments, compositions of the present invention comprise about 27% w/w of butylene glycol.

Compositions of the present invention may thus also comprise between about 0% w/w and about 80% w/w of pentylene glycol. In embodiments, compositions of the present invention comprise between about 20% w/w and about 80% w/w of pentylene glycol. In embodiments, compositions of the present invention comprise between about 0% w/w and about 22% w/w of pentylene glycol. In particular embodiments, between about 0% w/w and about 27.5% w/w of pentylene glycol. In embodiments, compositions of the present invention comprise between about 0% w/w and about 44% w/w of pentylene glycol. In embodiments, compositions of the present invention comprise between about 0% w/w and about 32% w/w of pentylene glycol. In embodiments, compositions of the present invention comprise between about 20% w/w and about 32% w/w of pentylene glycol. In embodiments, compositions of the present invention comprise between about 22% w/w and about 32% w/w of pentylene glycol. In embodiments, compositions of the present invention comprise between about 22% w/w and about 54% w/w of pentylene glycol. In embodiments, compositions of the present invention comprise between about 22% w/w and about 76% w/w of pentylene glycol. In embodiments, compositions of the present invention comprise between about 20% w/w and about 30% w/w of pentylene glycol. In particular embodiments, compositions of the present invention comprise about 22% w/w of pentylene glycol. In other particular embodiments, compositions of the present invention comprise about 44% w/w of pentylene glycol. In other particular embodiments, compositions of the present invention comprise about 76% w/w of pentylene glycol. In other particular embodiments, compositions of the present invention comprise about 27% w/w of pentylene glycol.

In particular embodiments, compositions of the present invention comprise about 2.5% ethoxydiglycol, and about 76% w/w of one or more of butylene glycol, propylene glycol and pentylene glycol. In particular embodiments, compositions of the present invention comprise about 2.5% w/w ethoxydiglycol, about 32% w/w of propylene glycol, about 22% w/w butylene glycol and about 22% w/w of pentylene glycol.

In other particular embodiments, compositions of the present invention comprise about 51% ethoxydiglycol, and about 27% w/w of one or more of butylene glycol, propylene glycol and pentylene glycol. In other particular embodiments, compositions of the present invention comprise about 51% ethoxydiglycol, and about 27% w/w of propylene glycol.

In embodiments compositions of the present invention further comprise, at least one antioxidant. In embodiments, the at least one antioxidant comprises a fruit extract, hydroquinone, vitamin E, a retinoid or any combination thereof. In embodiments, the at least one antioxidant comprises an antioxidant fruit extract. In embodiments the antioxidant fruit extract is a grapefruit extract. In embodiments, the compositions comprise about 1% w/w of grapefruit extract.

In particular embodiments, the pH of compositions of the present invention is between about 2 and about 4.

In embodiments, the above-noted compositions are (i) for treating or preventing skin inflammation, skin irritation, and/or skin's signs of aging; (ii) for promoting wound healing; (iii) for increasing skin thickness, reducing dryness or/or oiliness, improving skin texture, improving skin tone and preventing or reducing fine lines and wrinkles; and/or (iv) for preventing or reducing the formation of hypertrophic scar tissue.

In a related aspect, the present invention concerns the use of the above-noted topical composition (i) for treating or preventing skin inflammation, skin irritation, and/or skin's signs of aging; (ii) for promoting wound healing; (iii) for increasing skin thickness, reducing dryness or/or oiliness, improving skin texture, improving skin tone and preventing or reducing fine lines and wrinkles; and/or (iv) for preventing or reducing the formation of hypertrophic scar tissue.

In a related aspect, the present invention concerns a process for preparing/manufacturing a topical composition described herein.

In particular aspects, the present invention relates to the following items:
1. A slow release aqueous topical composition comprising (i) a primary polyamine; (ii) Vitamin C; (iii) ethoxydiglycol; and (iv) a glycol selected from (a) propylene glycol, (b) butylene glycol, (c) pentylene glycol, and (d) a combination of at least two of (a) to (d).
2. A slow-release aqueous topical composition comprising (i) at least 16% w/w of vitamin C; (ii) ethoxydiglycol; and (iii) a glycol selected from (a) propylene glycol, (b) butylene glycol, (c) pentylene glycol, and (d) a combination of at least two of (a) to (d).
3. A slow-release aqueous topical composition comprising (i) at least one active ingredient, (ii) ethoxydiglycol; (iii) a glycol selected from (a) propylene glycol, (b) butylene glycol, (c) pentylene glycol and (d) a combination of at least two of (a) to (d); and (iv) betaine and/or polysorbate 20.
4. The topical composition of item 3, comprising between about 0.5 w/w and about 3% w/w of betaine.
5. The topical composition of item 4, comprising about 1.2% of betaine.
6. The topical composition of any one of items 3 to 5, comprising between about 0.1% w/w and about 3% w/w of polysorbate 20.
7. The composition of item 6, comprising about 1.3% w/w of polysorbate 20.
8. The topical composition of any one of items 3 to 7, wherein the at least one active ingredient comprises a primary polyamine.
9. The topical composition of item 1 or 8, wherein said primary polyamine is a primary aliphatic lower-alkyl (C1-5) monoamine; a primary aliphatic alkylamine or a primary aliphatic lower-alkyl (C1-5) polyamine.
10. The topical composition of item 9, wherein said primary aliphatic lower-alkyl (C1-5) monoamine is aminoacetonitrile, said primary aliphatic alkylamine is spermine or spermidine and said primary aliphatic lower-alkyl (C1-5) polyamine is putrescine or dansylcadaverine.
11. The topical composition of item 10, comprising putrescine (1,4-diaminobutane).
12. The topical composition of item 11, comprising between about 0.1% w/w to about 1% w/w of putrescine.
13. The topical composition of item 12, comprising about 0.4% w/w of putrescine.
14. The topical composition of item 12, comprising about 0.8% w/w of putrescine.
15. The topical composition of any one of items 1 to 14, wherein the Vitamin C comprises L-ascorbic acid, 3-O-ethyl ascorbic acid (ETVC) or a combination thereof.
16. The topical composition of any one of items 1 and 3 to 14, wherein said composition comprises more than zero and up to about 20% w/w of 3-O-ethyl ascorbic acid, L-ascorbic acid or a combination thereof as Vitamin C.
17. The topical composition of item 16, comprising about 10% w/w of L-ascorbic acid and/or 3-O-ethyl ascorbic acid (ETVC).
18. The topical composition of item 16, comprising about 20% w/w of L-ascorbic acid and/or 3-O-ethyl ascorbic acid (ETVC).
19. The topical composition of item 17, comprising about 10% w/w of L-ascorbic acid.
20. The topical composition of item 17, comprising about 10% w/w of 3-O-ethyl ascorbic acid (ETVC).
21. The topical composition of item 16, comprising about 5% w/w of 3-O-ethyl ascorbic acid (ETVC).
22. The topical composition of item 16, comprising about 5% w/w of L-ascorbic acid and 5% w/w 3-O-ethyl ascorbic acid (ETVC).
23. The topical composition of item 16, comprising about 12.5% w/w of L-ascorbic acid and 7.5% w/w 3-O-ethyl ascorbic acid (ETVC).
24. The topical composition of item 16, comprising about 3% w/w of L-ascorbic acid and 7% w/w 3-O-ethyl ascorbic acid (ETVC).
25. The topical composition of any one of items 1 to 24, comprising between about 22% w/w and about 60% w/w of ethoxydiglycol.
26. The topical composition of item 25, comprising between about 48% w/w and about 55% w/w of ethoxydiglycol.
27. The topical composition of item 26, comprising about 49% w/w of ethoxydiglycol.
28. The topical composition of item 25, comprising about 43% w/w of ethoxydiglycol.
29. The topical composition of item 25, comprising about 33% w/w of ethoxydiglycol.
30. The topical composition of item 26, comprising about 51% w/w of ethoxydiglycol.
31. The topical composition of any one of items 1 to 30, comprising between about 20% w/w and about 30% w/w of propylene glycol.
32. The topical composition of item 31, comprising between about 26% w/w and about 27.5% w/w of propylene glycol.
33. The topical composition of item 31, comprising between about 23% w/w and about 27.5% w/w of propylene glycol.
34. The topical composition of item 31, comprising about 26% w/w of propylene glycol.
35. The topical composition of item 31, comprising about 27% w/w of propylene glycol.
36. The topical composition of item 31, comprising about 23% w/w of propylene glycol.
37. The topical composition of 1 to 24, comprising more than zero and up to 2.5% ethoxydiglycol.
38. The topical composition of item 37, comprising about 2.5% ethoxydiglycol.
39. The topical composition of item 38, wherein the glycol consists of a combination of (a) propylene glycol, (b) butylene glycol, and (c) pentylene glycol.
40. The topical composition of item 39, wherein the concentration of propylene glycol in the composition is about 32% w/w.
41. The topical composition of item 39 or 40, wherein the concentration of butylene glycol in the composition is about 22% w/w.
42. The topical composition of any one of items 39 to 41, wherein the concentration of pentylene glycol in the composition is about 22% w/w.
43. The topical composition of item 39, wherein the ratio of propylene glycol:butylene glycol:pentylene glycol is about 1.45:1:1 w/w.
44. The topical composition of any one of items 1 to 43, wherein the composition further comprises at least one antioxidant.
45. The topical composition of item 44, wherein at least one antioxidant comprises a fruit extract, hydroquinone, vitamin E, a retinoid or any combination thereof.
46. The topical composition of item 45, wherein at least one antioxidant comprises an antioxidant fruit extract.
47. The topical composition of item 46, wherein the antioxidant fruit extract is a grapefruit extract.
48. The topical composition of item 47, comprising about 1% of grapefruit extract.
49. The compositions of any one of items 1 to 48, wherein the pH of the composition is between about 2 and about 4.
50. The topical composition of any one of items 1 to 49, for use in treating or preventing skin inflammation, skin irritation, and/or skin's signs of aging such as thinning of skin, wrinkles and fine lines.
51. The topical composition of any one of items 1 to 49, for use in promoting wound healing.
52. The topical composition of any one of items 1 to 49, for use in preventing or reducing the formation of hypertrophic scar tissue.
53. The topical composition of any one of items 1 to 49, for use in (i) increasing the thickness of the skin; (ii) increasing skin tone or preventing loosening of the skin; (iii) reducing dryness of the skin; (iv) reducing oiliness of the skin; and/or (v) preventing or reducing the appearance of fine lines and wrinkles.
54. The topical composition for use of item 50 or 53, wherein the use is a cosmetic use.
55. Use of the topical composition as defined in any one of items 1 to 49, for treating or preventing skin inflammation, skin irritation, and/or skin's signs of aging.
56. Use of the topical composition as defined in any one of items 1 to 49, for promoting wound healing.
57. Use of the topical composition as defined in any one of items 1 to 49, for preventing or reducing the formation of hypertrophic scar tissue.
58. Use of the topical composition defined in any one of items 1 to 49, for (i) increasing the thickness of the skin; (ii) increasing skin tone or preventing loosening of the skin; (iii) reducing dryness of the skin; (iv) reducing oiliness of the skin; and/or (v) preventing or reducing the appearance of fine lines and wrinkles.
59. Use of the topical composition defined in any one of items 1 to 49, in the preparation of a cosmetic composition for use in (i) increasing the thickness of the skin; (ii) increasing skin tone or preventing loosening of the skin; (iii) reducing dryness of the skin; (iv) reducing oiliness of the skin; and/or (v) preventing or reducing the appearance of fine lines and wrinkles.

### BRIEF DESCRIPTION OF DRAWINGS

### In the Appended Drawings:

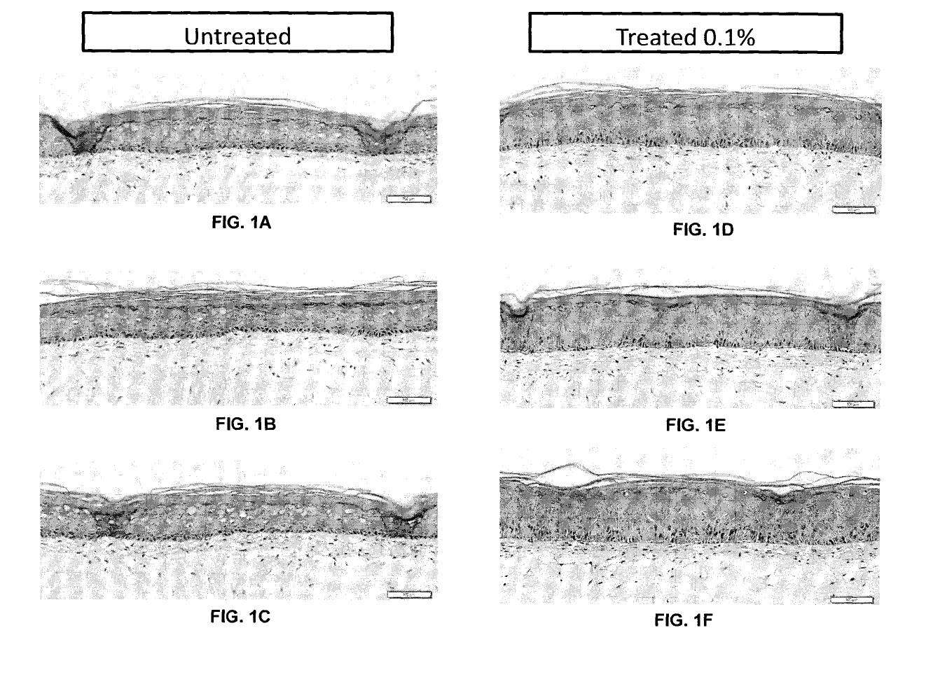

FIG. 1 shows exemplary Hematoxylin and Eosin staining of EFT-400 tissue sections treated or not with 0.1% Putrescine for 4 days. (A-C) Representative untreated tissue sections. (D-F) Representative tissues sections treated with 0.1% Putrescine for 4 days (see Example 16). Note the increased thickness of tissue sections treated with Putrescine. Rectangles located at the bottom left corners of FIGS. 1A-F represents a scale of 100 μm;

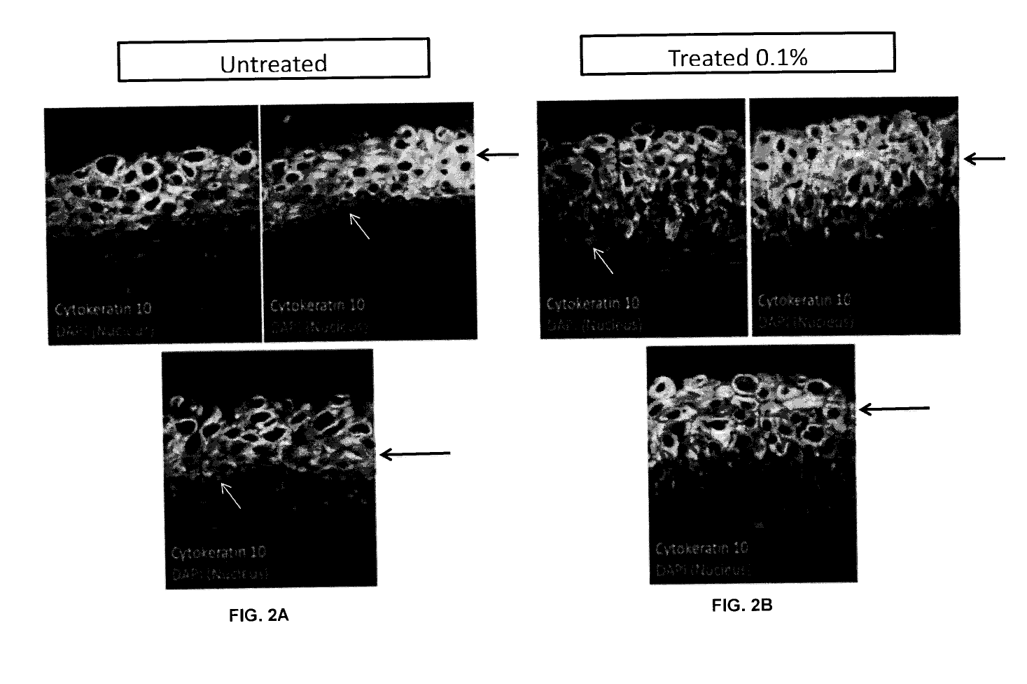

FIG. 2 shows immunofluorescence staining of EFT-400 tissue sections treated or not with 0.1% Putrescine. Tissues were stained for Cytokeratin 10 (thick arrows) and Desmoplakin-1 (DAPI, thin arrows);

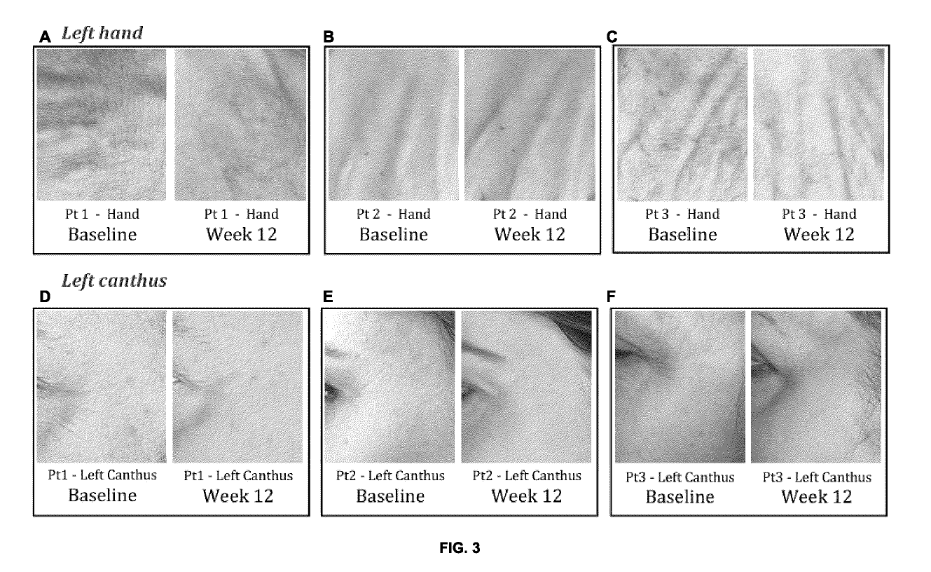

FIG. 3 shows that a composition comprising putrescine (0.4%) and L-Ascorbic acid (10%) improves the appearance of the skin following 12 weeks of treatment (twice daily). (A-C) Photographs of the top of the left hand of three (3) subjects at baseline (t=0) and after 12 weeks of treatment. (D-F) Photographs of the left Canthus of three (3) subjects at baseline (t=0) and after 12 weeks of treatment (see Examples 6 and 17); and

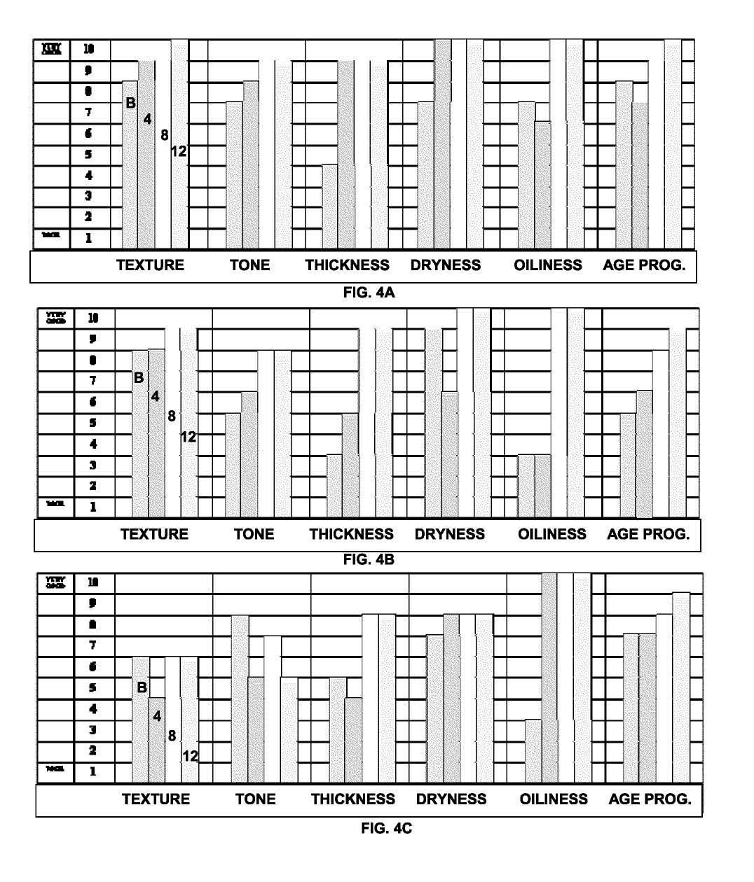

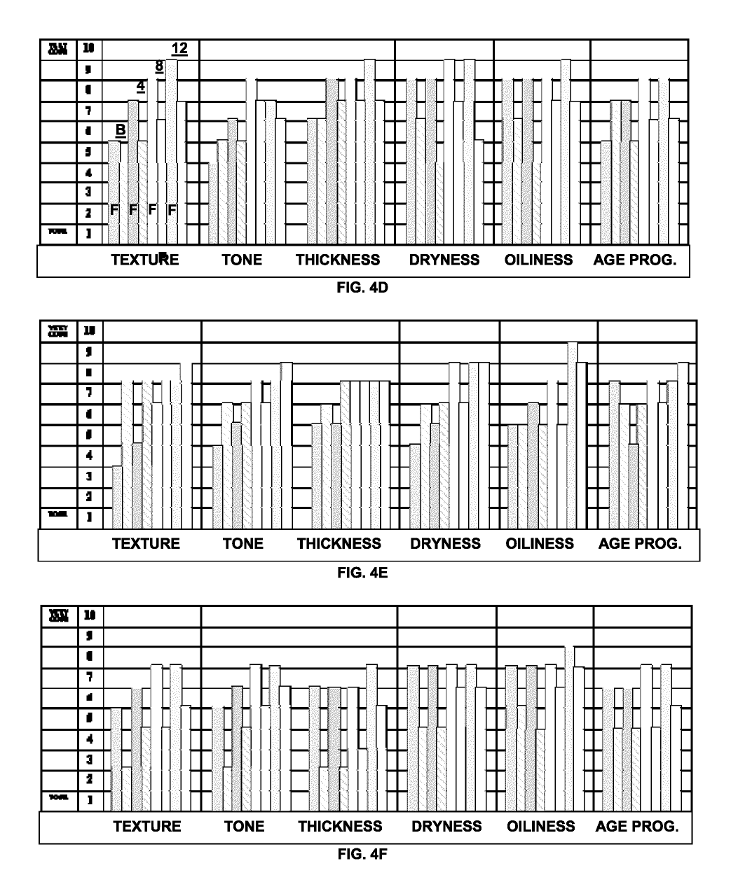

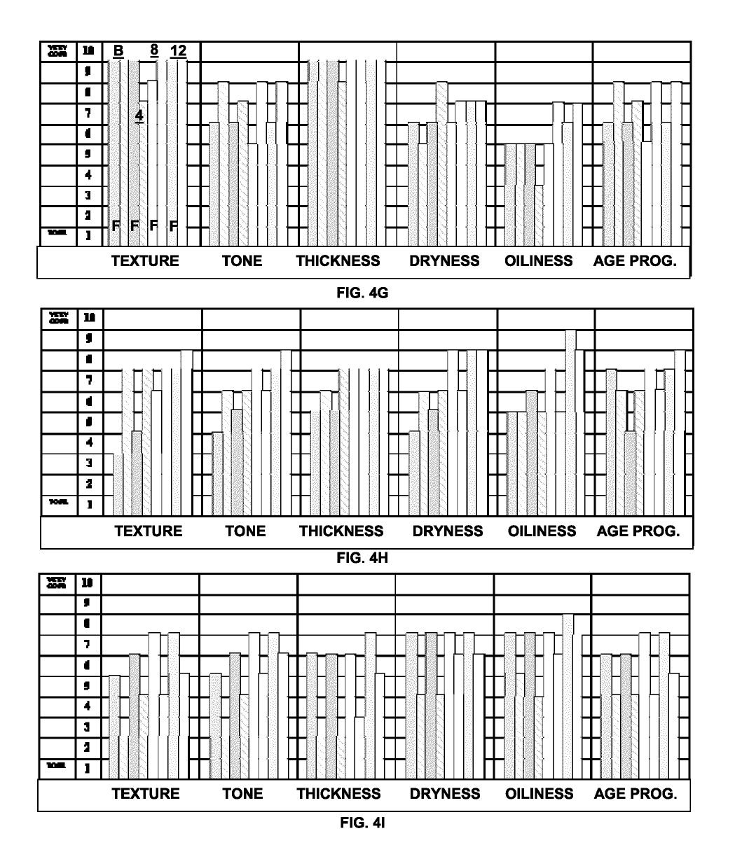

FIG. 4 shows improvements of skin texture, tone, thickness, dryness, oiliness and age progression over the 12 week-period of treatment in a cosmetic study. Bars for each criterion represent, from left to right: baseline (B), after 4 weeks (4), after 8 weeks (8), and after 12 weeks (12) of treatment. (A) Patient 1 assessment; (B) Patient 2 assessment; (C) Patient 3 assessment. (D) Investigator assessment for patient 1 (“F” for face); (E) Investigator assessment for patient 2; (F) Investigator assessment for patient 2; (G) Double blind randomized assessment for patient 1; (H) Double blind randomized assessment for patient 2; (I) Double blind randomized assessment for patient 3 (see Examples 6 and 17). In FIGs. D-I, the first bar represents face treatment (identified as “F” on FIGs. D and G) and the next bar (unlabeled) represents hand treatment.

### DESCRIPTION OF ILLUSTRATIVE EMBODIMENTS

Not all cosmetic products are alike. Important factors affecting cosmetic and therapeutic results include the stability of the active ingredient(s) in the compositions and their ability to penetrate the skin and reach its targeted layer(s) (e.g., the dermis and/or the epidermis). Applicants have developed new aqueous topical slow-release compositions in which the active ingredients (e.g., putrescine and derivatives thereof, Vitamin C and derivatives thereof and/or other antioxidants) i) are stable; and 2) efficiently penetrate the skin, thereby enabling the active ingredients to reach the cells and provide the desired effect.

In a first aspect, the present invention provides a slow-release topical formulation comprising (i) at least one active ingredient (e.g., a primary polyamine and/or vitamin C); (ii) ethoxydiglycol; (iii) propylene glycol; and (iv) (a) betaine and/or (b) polysorbate 60 and/or polysorbate 20 (preferably polysorbate 20). In embodiments, the active ingredient comprises Vitamin C and/or a primary polyamine.

In a further aspect, the present invention provides a slow-release topical formulation comprising (i) at least one primary polyamine (e.g., putrescine, spermine, spermidine, cadaverine, dansylcadaverine, aminoacetonitrile and derivatives thereof); (ii) ethoxydiglycol; and (iii) propylene glycol, butylene glycol, pentylene glycol, or a mixture of at least two of propylene glycol, butylene glycol and pentylene glycol. In embodiments, the formulation includes butylene glycol, propylene glycol and pentylene glycol. In embodiments, the formulation only includes propylene glycol and ethoxydiglycol as glycols. In embodiments, the formulation further advantageously includes (iv) (a) betaine and/or (b) polysorbate 60 and/or polysorbate 20 (preferably polysorbate 20).

In another aspect, the present invention provides a slow-release topical formulation comprising (i) at least 16%, of Vitamin C, (ii) ethoxydiglycol; and (iii) propylene glycol, butylene glycol, pentylene glycol, or a mixture of at least two of propylene glycol, butylene glycol and pentylene glycol. In embodiments, the formulation includes butylene glycol, propylene glycol and pentylene glycol. In embodiments, the formulation only includes propylene glycol and ethoxydiglycol as glycols. In some embodiments, the formulation further advantageously includes (iv) (a) betaine and/or (b) polysorbate 60 and/or polysorbate 20 (preferably polysorbate 20).

In another aspect, the present invention provides a slow-release topical formulation comprising at least one polyamine (e.g., putrescine, spermine, cadaverine and derivatives thereof); (ii) Vitamin C; (iii) ethoxydiglycol; and (iv) propylene glycol, butylene glycol, pentylene glycol, or a mixture of at least two of propylene glycol, butylene glycol and pentylene glycol. In embodiments, the formulation includes butylene glycol, propylene glycol and pentylene glycol. In embodiments, the formulation only includes propylene glycol and ethoxydiglycol as glycols. In embodiments some embodiments, the formulation further advantageously includes (v) (a) betaine and/or (b) polysorbate 60 and/or polysorbate 20 (preferably polysorbate 20).

Topical formulations of the present invention were found to be stable and enable the effective penetration and delivery of active ingredients such as polyamines (e.g., putrescine) and Vitamin C (i.e., ascorbic acid and derivatives thereof) into the skin.

The primary polyamines used in accordance with the present invention are preferably amine group terminated linear structures such as unbranched aliphatic compounds (e.g., lower C1-C10, preferably, C1-C5 alkyls). Such compounds include, but are not limited to naturally occurring putrescine (1,4-diaminobutane (Cas #333-93-7), H2N(CH2)4NH2), cadaverine (Cas#462-94-2, 1,5-pentanediamine, H2N(CH2)5NH2), spermidine (Cas#124-20-9, 1,4-butanediamine, N1-(3-aminopropyl, H2N(CH2)3NH(CH2)4NH2), spermine (Cas #71-44-3, 1,4-Butanediamine, N,N′-bis(3-aminopropyl), H₂N(CH₂)₃NH(CH₂)₄NH(CH₂)₃NH₂) and their functional derivatives. The polyamines preferably have (CH₂)ₙ groups linking the nitrogens where n is 2 to 10, preferably 2 to 6, more preferably 2 to 5 and particularly ones comprising 2 to 6 nitrogens, particularly 2, 3 or 4 nitrogens. These polyamines are available from natural sources, e.g. mammalian semen or fermentation products (for example from soy or anchovies), or may be manufactured by conventional techniques, e.g. solid state polypeptide production followed by amidation and reduction. Polyamines useful in accordance with the present invention are described for example in WO2006/048671, U.S. Pat. No. 5,885,982 and CA 2,706,630 The polyamine(s) used in accordance with the invention may conveniently be in salt form with a physiologically tolerable counterion, (e.g. inorganic/mineral acid, an organic acid such as an alpha-hydroxyacid or a fatty acid). Such salts, may be prepared by reaction of the polyamine and the acid, e.g. in solution for example in approximately equimolar amounts.

Under certain aspects, the total polyamine content in the compositions of the present invention is between about 0.0005 and about 5% w/w (e.g., between about 0.001% w/w and about 1% w/w, between about 0.005% w/w and about 1% w/w, between about 0.1% w/w and about 1% w/w.). Preferably, in compositions for use in accordance with the present invention (e.g., reducing the appearance of scar tissue, including hypertrophic scar tissue, promoting skin thickening and reducing signs of aging (e.g., fine lines and wrinkles, sagginess, etc.), the concentration of putrescine is preferably between about 0.1% w/w and about 1% w/w, more preferably between about 0.4% w/w and about 0.8% w/w.

In certain aspects, compositions of the present invention comprise Vitamin C. As used herein, the term “vitamin C” refers to ascorbic acid and its derivatives. Non-limiting examples of suitable forms of Vitamin C include: L-ascorbic acid, sodium ascorbyl phosphate (SAP), magnesium ascorbyl phosphate (MAP), ascorbyl palmitate (AA-PAL), ascorbyl tetra-isopalmitate (VC-IP), ascorbyl glucoside (AA-2G), ascorbyl 2-phosphate 6-palmitate (AAPS), 3-O-ethylascorbate (EAC), 3-O-ethyl ascorbic acid (e.g., ET-VC™). Preferably, compositions of the present invention comprise 3-O-ethyl ascorbic acid, ascorbyl palmitate and/or L-ascorbic acid, more preferably, ethyl ascorbic acid and/or L-ascorbic acid.

Under certain aspects, the concentration of Vitamin C in compositions of the present invention is between about 0.01% w/w and about 40% w/w, preferably between about 0.1% w/w and about 35% w/w, more preferably between about 1% w/w and about 30% w/w. In embodiments, compositions of the present invention comprise at least 12% w/w, at least 15% w/w, at least 18% w/w at least 20% w/w of Vitamin C. In embodiments, the concentration of Vitamin C is up to 30%. In embodiments, the concentration of Vitamin C is about 0.5% w/w, about 1% w/w, about 5% w/w, about 10% w/w, about 12%, about 12.5%, about 8%, about 15% w/w, about 16%, about 18% w/w, about 20% w/w, about 25% w/w or about 30% w/w.

Compositions of the present invention also comprise ethoxydiglycol. The concentration of ethoxydiglycol in the composition is generally between about between 0.1% w/w and about 60% w/w. In embodiments, the concentration of ethoxydiglycol is at least 2.5%. Preferably, the concentration of ethoxydiglycol is between about 45% w/w and about 55%, more preferably, between about 48% w/w and 55% w/w (48%, 48.1%, 48.2%, 48.3%, 48.4%, 48.5%, 48.6, 48.7%, 48.8%, 48.9%, 50%, 50.1%, 50.2%, 50.3%, 53.4%, 50.5%, 50.6%, 50.7%, 50.8%, 50.9%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55% w/w). In embodiments, the concentration of ethoxydiglycol is about 49.5% w/w. In embodiments, the concentration of ethoxydiglycol is about 49.8% w/w. In embodiments, the concentration of ethoxydiglycol is about 51.3% w/w. In embodiments, the concentration of ethoxydiglycol is about 2.5% w/w.

Compositions of the present invention also comprise one or more additional glycol (e.g., propylene glycol, butylene glycol and/or pentylene glycol). In compositions comprising low concentrations of ethoxydiglycol (e.g., up to 2.5% ethoxydiglycol), the concentration of other glycols (e.g., propylene glycol, butylene glycol and/or pentylene glycol) is generally up to about 80% w/w of the composition.

Accordingly, compositions of the present invention also comprise propylene glycol, butylene glycol and/or pentylene glycol.

In certain aspects, the concentration of propylene glycol in the composition is generally between about between 0.1% w/w and about 80% w/w. In certain aspects, the concentration of propylene glycol in the composition is between about between 0.1% w/w and about 30% w/w. Preferably, the concentration of propylene glycol is between about 17% w/w and about 30%, more preferably, between about 17% w/w and about 32% w/w, (17%, 17.1%, 17.2%, 17.3%, 17.4%, 17.5%, 17.6, 17.7%, 17.8%, 17.9%, 18%, 18.1%, 18.2%, 18.3%, 18.4%, 18.5%, 18.6, 18.7%, 18.8%, 18.9%, 19%, 19.1%, 19.2%, 19.3%, 19.4%, 19.5%, 19.6, 19.7%, 19.8%, 19.9%, 20%, 20.1%, 20.2%, 20.3%, 20.4%, 20.5%, 20.6, 20.7%, 20.8%, 20.9%, 21%, 21.1%, 21.2%, 21.3%, 21.4%, 21.5%, 21.6, 21.7%, 21.8%, 21.9%, 22%, 22.1%, 22.2%, 22.3%, 22.4%, 22.5%, 22.6, 22.7%, 22.8%, 22.9%, 23%, 23.1%, 23.2%, 23.3%, 53.4%, 23.5%, 23.6%, 23.7%, 23.8%, 23.9%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.1%, 30.20%, 30.30%, 30.40%, 30.50%, 30.60%, 30.70%, 30.80%, 30.90%, 31.00%, 31.10%, 31.20%, 31.30%, 31.40%, 31.50%, 31.60%, 31.70%, 31.80%, 31.90%, 32.00% w/w). In embodiments, the concentration of propylene glycol is between about 25% w/w and 28% w/w. In embodiments, the concentration of propylene glycol is about 27.1% w/w. In embodiments, the concentration of propylene glycol is about 26% w/w. In embodiments, the concentration of propylene glycol is about 31.9% w/w.

In certain aspects, the concentration of butylene glycol in the composition is generally between about between 0.1% w/w and about 80% w/w. Preferably, the concentration of butylene glycol is between about 0.1% w/w and about 45%, more preferably, between about 20% w/w and about 45% w/w (20%, 20.1%, 20.2%, 20.3%, 20.4%, 20.5%, 20.6, 20.7%, 20.8%, 20.9%, 21%, 21.1%, 21.2%, 21.3%, 21.4%, 21.5%, 21.6, 21.7%, 21.8%, 21.9%, 22%, 22.1%, 22.2%, 22.3%, 22.4%, 22.5%, 22.6, 22.7%, 22.8%, 22.9%, 23%, 23.1%, 23.2%, 23.3%, 53.4%, 23.5%, 23.6%, 23.7%, 23.8%, 23.9%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.10%, 30.20%, 30.30%, 30.40%, 30.50%, 30.60%, 30.70%, 30.80%, 30.90%, 31.00%, 31.10%, 31.20%, 31.30%, 31.40%, 31.50%, 31.60%, 31.70%, 31.80%, 31.90%, 32.00%, 32.10%, 32.20%, 32.30%, 32.40%, 32.50%, 32.60%, 32.70%, 32.80%, 32.90%, 33.00%, 33.10%, 33.20%, 33.30%, 33.40%, 33.50%, 33.60%, 33.70%, 33.80%, 33.90%, 34.00%, 34.10%, 34.20%, 34.30%, 34.40%, 34.50%, 34.60%, 34.70%, 34.80%, 34.90%, 35.00%, 35.10%, 35.20%, 35.30%, 35.40%, 35.50%, 35.60%, 35.70%, 35.80%, 35.90%, 36.00%, 36.10%, 36.20%, 36.30%, 36.40%, 36.50%, 36.60%, 36.70%, 36.80%, 36.90%, 37.00%, 37.10%, 37.20%, 37.30%, 37.40%, 37.50%, 37.60%, 37.70%, 37.80%, 37.90%, 38.00%, 38.10%, 38.20%, 38.30%, 38.40%, 38.50%, 38.60%, 38.70%, 38.80%, 38.90%, 39.00%, 39.10%, 39.20%, 39.30%, 39.40%, 39.50%, 39.60%, 39.70%, 39.80%, 39.90%, 40.00%, 40.10%, 40.20%, 40.30%, 40.40%, 40.50%, 40.60%, 40.70%, 40.80%, 40.90%, 41.00%, 41.10%, 41.20%, 41.30%, 41.40%, 41.50%, 41.60%, 41.70%, 41.80%, 41.90%, 42.00%, 42.10%, 42.20%, 42.30%, 42.40%, 42.50%, 42.60%, 42.70%, 42.80%, 42.90%, 43.00%, 43.10%, 43.20%, 43.30%, 43.40%, 43.50%, 43.60%, 43.70%, 43.80%, 43.90%, 44.00%, 44.10%, 44.20%, 44.30%, 44.40%, 44.50%, 44.60%, 44.70%, 44.80%, 44.90%, 45.00%, w/w). In embodiments, the concentration of butylene glycol is between about 22% w/w and 44% w/w. In embodiments, the concentration of butylene glycol is about 22% w/w.

In certain aspects, the concentration of pentylene glycol in the composition is generally between about between 0.1% w/w and about 80% w/w. Preferably, the concentration of pentylene glycol is between about 0.1% w/w and about 45%, more preferably, between about 20% w/w and about 45% w/w (20%, 20.1%, 20.2%, 20.3%, 20.4%, 20.5%, 20.6, 20.7%, 20.8%, 20.9%, 21%, 21.1%, 21.2%, 21.3%, 21.4%, 21.5%, 21.6, 21.7%, 21.8%, 21.9%, 22%, 22.1%, 22.2%, 22.3%, 22.4%, 22.5%, 22.6, 22.7%, 22.8%, 22.9%, 23%, 23.1%, 23.2%, 23.3%, 53.4%, 23.5%, 23.6%, 23.7%, 23.8%, 23.9%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.10%, 30.20%, 30.30%, 30.40%, 30.50%, 30.60%, 30.70%, 30.80%, 30.90%, 31.00%, 31.10%, 31.20%, 31.30%, 31.40%, 31.50%, 31.60%, 31.70%, 31.80%, 31.90%, 32.00%, 32.10%, 32.20%, 32.30%, 32.40%, 32.50%, 32.60%, 32.70%, 32.80%, 32.90%, 33.00%, 33.10%, 33.20%, 33.30%, 33.40%, 33.50%, 33.60%, 33.70%, 33.80%, 33.90%, 34.00%, 34.10%, 34.20%, 34.30%, 34.40%, 34.50%, 34.60%, 34.70%, 34.80%, 34.90%, 35.00%, 35.10%, 35.20%, 35.30%, 35.40%, 35.50%, 35.60%, 35.70%, 35.80%, 35.90%, 36.00%, 36.10%, 36.20%, 36.30%, 36.40%, 36.50%, 36.60%, 36.70%, 36.80%, 36.90%, 37.00%, 37.10%, 37.20%, 37.30%, 37.40%, 37.50%, 37.60%, 37.70%, 37.80%, 37.90%, 38.00%, 38.10%, 38.20%, 38.30%, 38.40%, 38.50%, 38.60%, 38.70%, 38.80%, 38.90%, 39.00%, 39.10%, 39.20%, 39.30%, 39.40%, 39.50%, 39.60%, 39.70%, 39.80%, 39.90%, 40.00%, 40.10%, 40.20%, 40.30%, 40.40%, 40.50%, 40.60%, 40.70%, 40.80%, 40.90%, 41.00%, 41.10%, 41.20%, 41.30%, 41.40%, 41.50%, 41.60%, 41.70%, 41.80%, 41.90%, 42.00%, 42.10%, 42.20%, 42.30%, 42.40%, 42.50%, 42.60%, 42.70%, 42.80%, 42.90%, 43.00%, 43.10%, 43.20%, 43.30%, 43.40%, 43.50%, 43.60%, 43.70%, 43.80%, 43.90%, 44.00%, 44.10%, 44.20%, 44.30%, 44.40%, 44.50%, 44.60%, 44.70%, 44.80%, 44.90%, 45.00%, w/w). In embodiments, the concentration of pentylene glycol is between about 22% w/w and 44% w/w. In embodiments, the concentration of pentylene glycol is about 22% w/w.

In embodiments, compositions of the present invention preferably comprise polysorbate 60, polysorbate 20 and/or betaine. The concentration of polysorbate 20 and/or 60 in compositions of the present invention is between about 0.1 w/w and about 3% w/w. Preferably, the concentration of polysorbate 20 and/or 60 is between about 1% and about 2% (between about 1.1% and about 2%, between about 1.2% and about 2%, between about 1.3% and about 2%, between about 1.1% and about 1.5%, between about 1.2% and about 1.5%, etc.). In embodiments, the concentration of polysorbate 20 and/or 60 is about 1.3% w/w. Preferably, compositions of the present invention uses polysorbate 20 because applicants have found that it allows to improve stability of compositions of the present invention. The concentration of betaine in compositions of the present invention is between about 0.5% w/w and about 3% w/w, preferably between about 1% w/w and 2% w/w, more preferably between about 1% w/w and about 1.5% w/w (1%, 1.1%, 1.2%, 1.3%, 1.4%, 1.5% w/w). In embodiments, the concentration of betaine is about 1.3% w/w.

Compositions of the present invention may additionally comprise one or more active ingredients (e.g., useful for reducing or preventing skin aging, skin irritation and inflammation, for improving skin texture, skin tone, skin thickness and/or skin healing). As used herein, the term “active ingredient” refers to various types of optional additional active ingredients that may be used in compositions of the present invention. Actives are defined as skin benefit agents other than emollients and ingredients that merely improve the physical characteristics of the composition. Although not limited to this category, common examples include talcs and silicas, zinc salts, and sunscreens.

Non-limiting examples of active ingredients that may be added in compositions of the present invention include: retinol, Pseudoalteromonas ferment extract, hydrolyzed wheat protein, hydrolyzed soy protein, glycine soja (soybean) protein, citrulline, tripeptide-1 (glycine,-histidine-lysine), tripeptide-5, palmitoyl tripeptide-5, tripeptide-8, tripeptide-10, glycine, Butyrospermum parkii (shea) butter, argania spinosa kernel oil, jojoba esters, glucose, hydrolyzed rice protein, superoxide dismutase, Rosmarinus officinalis (rosemary) leaf extract, cetearyl olivate, sorbitan olivate, Ruscus aculeatus root extract, Centella asiatica extract, hydrolyzed yeast protein, hydrolyzed casein, Calendula officinalis flower extract, acetyl hexapeptide-3, allantoin, citrus grandis (grapefruit) extract, hydrolyzed glycosaminoglycans, hyaluronic acid, acetylated hyaluronic acid, sodium hyaluronate, Persea gratissima (avocado) oil, tropolone, lysine hcl, Porphyridium cruentum extract, dimethiconol, caprylic/capric triglyceride, Cytokinol™, phytonadione (Vitamin K), Vitamin E (tocopherols (e.g., γ-tocopherol) and tocotrienols), escin, panthenol, Argireline, Kinetine, CE ferulic Acid, skin growth factors, Petrolatum/Canolin, dimethyl sulphoxide, coconut oil, keratolytic agents, unsaturated fatty acids (e.g. omega-3, omega-6 and omega-9 unsaturated fatty acids, especially omega-3 acids, for example EPA, DHA and ALA) and derivatives (particularly esters) thereof, HMG-CoA reductase inhibitors, natural triterpenes, Coenzyme Q10 (ubiquinone), vitamin B3, hydroquinone (tocopheryl acetate), Aloe, Mallotus japonicus extract, hydroxyacids (e.g. alpha hydroxy acids such as glycolic acid), beta-(l,3) glucans, extract of unpolished rice, urea, pine seed oil, marine collagens, plant cell extracts, ceramides, cholesterol, glutathione, carnitine, caffeine, Rosa mosqueta oil, cystein derivatives, acid and alpha-amino acids, and salts of any of these.

In embodiments, compositions of the present invention comprise one or more antioxidants. As used herein, the term “antioxidant” refers to compounds, natural or synthetic, capable of neutralizing reactive oxygen species (ROS). Commonly used antioxidants in compositions (dermatological compositions) include, for example, ascorbic acid (Vitamin C), tocopherol (Vitamin E), isoflavones, polyphenols, and retinoids, (including retinoic acid (0.25% to 0.1%), tretinoin, retinal, retinol (0.1% to 5%), Adapalene, tazorotene and retinyl esters. Reviewed in Sheri L. Rolewski. Dermatology Nursing. 2003; 15(5), Jannetti Publications, Inc.), alpha lipoic acid, beta-glucan, coenzyme Q10, grape seed extract, amino acids, green tea, soybean sterols, ergothioneine (EGT, a thiourea derivative of histidine), Resorcinol, Carcinine and mixtures thereof. In embodiments, compositions of the present invention comprise Vitamin C and at least one further antioxidant. In embodiments, compositions of the present invention further comprise (in addition to putrescine and/or Vitamin C) one or more of the following active ingredients: an antioxidant (e.g., a retinoid such as retinol), grapefruit extract, Vitamin E and/or hydroquinone. Generally, the concentration of retinoids (such as retinol) that may be used in accordance with the present invention is between about 0.01% and 5%.

Generally the total amount of active ingredients in compositions of the present invention may be up to 40% w/w of the composition. In embodiments, the total amount of active ingredients in compositions of the present invention is between about 0.4% w/w and about 35% w/w. In embodiments, the total amount of active ingredients is between about 0.4% and about 30%. In embodiments, the total amount of active ingredients is up to 25% w/w of the composition. In embodiments, the total amount of active ingredients is up to 20% w/w of the composition.

The compositions according to the invention may be in any form suitable for topical application, e.g. creams, lotions, ointments, gels, balm, solutions, emulsions, dispersions, suspensions etc. and may if desired include a carrier substrate, e.g. a woven or nonwoven web. The compositions may contain conventional topical composition components, such as for example, solvents, oils (e.g. plant oils), aromas, sunscreens, colorants, pH modifiers, viscosity modifiers, binders, diluents, emollients, skin irritants, thickeners, preservatives, stabilizers, humidifiers, skin penetration enhancers, vesicle wall formers, etc. Preferably, compositions of the present invention are topical serums, creams, ointments.

Sunscreens include those materials commonly employed to block ultra-violet radiation. Illustrative compounds are the derivatives of para-aminobenzoic acid (PABA), cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trade-marks, Parsol MCX™, Parsol HS and Benzophenone-3™, respectively. The exact amount of sunscreen employed in the compositions can vary depending upon the degree of protection desired from the sun's ultra-violet radiation. Additives that reflect or scatter the sun rays may also be employed. These additives include oxides like zinc oxide and titanium dioxide.

Non-limiting examples of conventional topical composition components that may be included in compositions of the present invention include: lecithin, xanthan gum, carbomer, triethanolamine, phenoxyethanol, butylene glycol, caprylyl glycol, glyceryl stearate, PEG-100 stearate, PEG-75 stearate, PEG 40, dimethicone, glycerin, behenyl alcohol, cetyl palmitate, cyclopentasiloxane, dimethiconol, acrylates/acrylamide copolymer, magnesium aluminum silicate, methylparaben, ethylparaben, propylparaben, butylparaben, stearic acid, caprylic/capric triglyceride, titanium dioxide, triethoxycaprylylsilane, castor oil phosphate, tocopheryl acetate, tetrasodium edta, butylated hydroxy toluene, allyl methacrylates crosspolymer, polysorbate 20, carrageenan (chondrus crispus), ethylhexylglycerin, cetyl alcohol, ceteth-20, steareth-20, pentylene glycol, sodium benzoate, sodium dextran sulfate, potassium sorbate, ammonium glycyrrhizate, ethoxydiglycol, propylene glycol, betaine, saccharide isomerate, trimethylolpropane, tricaprylate/tricaprate, cetyl alcohol, dmdm hydantoin, isobutylparaben, 1,2-hexanediol, 1,2-octanediol, hydrogenated palm glycerides, glyceryl polyacrylate, mineral oil, allyl methacrylate crosspolymer, polysorbate-85, glyceryl dilaurate, C13-14 isoparaffin, laureth-7, C12-13 pareth-23, Hexamidine Diisethionate, Petrolatum & derivatives, Benzoyl Peroxide, Lanolin.

Many compositions, especially those containing water, may be protected against the growth of potentially harmful microorganisms. Anti-microbial compounds and preservatives are, therefore, typically incorporated into such compositions. Suitable preservatives include alkyl esters of p-hydroxybenzoic acid (parabens), hydantoin derivatives, propionate salts, parabens and a variety of quaternary ammonium compounds as well as chelating agents such as EDTA and well known non-parabens antimicrobial of all kinds.

Compositions of the present invention are water based (aqueous) formulations preferably having an acidic pH (i.e., below 7). Compositions of the present invention comprising Vitamin C preferably have an acidic pH which is below the pKa of ascorbic acid (around 4.17) when this pH is compatible with the stability of the other active ingredients in the composition (e.g., between about 2 and 3.2). Similarly, compositions comprising putrescine should have a pH below its pKa (which is 10.51). At pH below 4.2, putrescine and ascorbic acid are both in their active forms. In embodiments, slow-release compositions comprising about 10% w/w of Vitamin C and about 0.4% to about 0.8% w/w of putrescine have a pH of about 2.5 to about 3. Preferably, the water content of compositions of the present invention is between 0.1% and 30%.

### Uses

Compositions of the present invention are intended to be used as is, or through the making of a composition or a medication, to prevent or to treat any skin condition that involves or is caused by ROS or involving collagen synthesis or transglutaminase activity. The skin condition includes but is not limited to skin irritation or inflammation, dermatitis, skin allergy, psoriasis, acne, eczema, rosacea, radiations exposure including U.V. and/or sun exposure, laser exposure, skin aging (e.g., appearance of wrinkles, sagginess, thinning of the skin, etc), dry skin, skin surgery, wound healing. Compositions comprising putrescine (1,4-diaminobutane) and preferably putrescine and Vitamin C are particularly useful for promoting skin thickening (resulting in reduced fined lines and wrinkles), promoting wound healing and preventing and/or treating scars including hypertrophic scar tissue.

### General Manufacturing Procedures

Compositions of to the invention may be produced by standard cosmetic or pharmaceutical composition production techniques.

However, the following process has been found particularly useful in preparing compositions of the present invention.

The solution is mixed until clear after every addition. All steps, except the heating step, if required, are performed at room temperature (18-25° C.).

For compositions comprising Vitamin C, the process comprises the step of dissolving a first quantity of ascorbic acid in water (e.g., about 10%), which is followed by addition of a first quantity of ethoxydiglycol, under high stirring speed. The step of adding ascorbic acid and ethoxydiglycol (and/or other glycols) can be repeated numerous times (at least one other time), depending on the concentration of ascorbic acid that is sought. After sequential additions of second or third quantities of ascorbic acid and ethoxydiglycol, propylene glycol is added to contribute, with water, in the solubilization of ascorbic acid. The rapid agitation and the presence of ethoxydiglycol after each sequential addition of ascorbic acid provide for a micronized and stabilized solution.

Generally compositions of the present invention comprising above 10% of Vitamin C are preferably prepared by adding Vitamin C in multiple steps. Furthermore, when concentrations of ascorbic acid of about 6% or more are needed, a mild heating step (at least about 37-42° C., preferably 40° C.) is required to solubilize the last added quantities of Vitamin C, in the presence of propylene glycol (and/or butylene glycol and/or pentylene glycol). For formulating Vitamin C concentration of 15% or more, the concentration of water is increased from 10% to 15% or more. In such a case, sequential additions of ascorbic acid and ethoxydiglycol (and optionally other glycols such as propylene glycol, butylene glycol and/or pentylene glycol) may enable achieving a higher concentration of Vitamin C (12% again in the presence of propylene glycol, pentylene glycol and/or butylene glycol) prior to heating. A last addition of Vitamin C can be made, followed by a last heating step. Upon cooling at room temperature, the solution remains stable.

These steps (together with the other ingredients in the formulation) help to dissolve high amounts of Vitamin C and formulate stable, homogeneous slow-release compositions comprised of high amounts of water (e.g., 8-20%). Of note, Vitamin C is known to be heat sensitive and to start oxidizing at moderate temperature (e.g., at 35-40 degrees Celsius), in aqueous, especially acidic environments. However, the presence of ethoxydiglycol and other glycol(s) (propylene glycol, butylene glycol and/or pentylene glycol) is thought to provide protection against oxidative damage in acidic aqueous formulations and contribute to the stability of the formulation. Furthermore, the manufacturing procedure is generally performed under a nitrogen blanket or an equivalent procedure to reduce the possibility of oxidative damages to the composition.

For compositions comprising putrescine, the putrescine is preferably added at the end of the process, after ascorbic acid and other optional ingredients (e.g., betaine and polysorbate 20 or 60) have been added, when the solution is cooled.

The selection of the right solvents which involves particularly a high concentration of glycols, (ethoxydiglycol and propylene glycol, butylene glycol and/or pentylene glycol) and of the right sequential additions of ascorbic acid, putrescine, glycol(s) (e.g., ethoxydiglycol, propylene glycol, butylene glycol and/or pentylene glycol) and in some embodiments betaine and polysorbate (20 and/or 60), combined to a high speed that allows micronization of active ingredients (e.g., ascorbic acid) into such a solution, all contribute to obtaining a stable solution of ascorbic acid and/or putrescine. A micronization process appears to result in a product wherein the contact of oxygen with the vitamin, is sharply reduced once the latter is in solution. This reduces the oxidative damages to the Vitamin C. The process provides for a solution that has a shelf life of at least about three (3) years, without any substantial development of yellowish color neither affecting pH and concentration.

Without being bound to any particular theory, the deep penetrating and slow-release properties of the compositions of the present invention (e.g., comprising putrescine and/or Vitamin C) are thought to be due to hydrophilic and lipophilic activities of the components interacting together.

Other objects, advantages and features of the present invention will become more apparent upon reading the following non-restrictive description of specific embodiments thereof, given by way of example only with reference to the accompanying drawings.

The present invention is illustrated in further details by the following non-limiting examples.

### Example 1

### Deep Penetrating and Slow-Release Basic Formulation

TABLE 1
Basic deep penetrating, slow-release composition

| | | | | Amount |
|---|---|---|---|---|
| | Ingredients | CAS# | Grade | (% W/W) |
| 1 | Purified water | N.A. | USP | 20.4 |
| 2 | Citrus Grandis | 90045-43-5, | MFR | 1.0 |
| | Grapefruit Extract | 57-55-6 | | |
| | Antioxydant | | | |
| | (Greentech) | | | |
| 3 | Propylene Glycol | 57-55-6 | USP | 26.0 |
| 4 | Ethoxydiglycol | 111-90-0 | USP | 49.80 |
| | (Transcutol CG) | | | |
| 5 | Polysorbate 60 | 9005-67-8 | MFR | 1.30 |
| 6 | Betaine | 107-43-7 | MFR | 1.20 |
| 7 | Fragrance (Apple | | MFR | 0.30 |
| | Crunch #234-795) | | | |
| | | | | 100% |

The above formulation was prepared by adding, in a suitable stainless steel tank equipped with a lightening type propeller mixer, all ingredients one by one in the order they appear in Table 1 until the composition becomes clear and all ingredients have dissolved. A nitrogen blanket was used throughout the entire manufacturing process.

### Example 2

### Putrescine Deep Penetrating and Slow-Release Composition

TABLE 2
Exemplary 0.8% putrescine deep penetrating and slow-release composition

| | | | | Amount |
|---|---|---|---|---|
| | Ingredients | CAS# | Grade | (% W/W) |
| 1 | Purified water | N.A. | USP | 20% |
| 2 | Citrus Grandis | 90045-43-5, | MFR | 1.0 |
| | Grapefruit Extract | 57-55-6 | | |
| | Antioxydant | | | |
| | (Greentech) | | | |
| 3 | Propylene Glycol | 57-55-6 | USP | 26.0 |
| 4 | Ethoxydiglycol | 111-90-0 | USP | 49.5 |
| | (Transcutol CG) | | | |
| 5 | Polysorbate 60 | 9005-67-8 | MFR | 1.30 |
| 6 | Betaine | 107-43-7 | MFR | 1.20 |
| 7 | Fragrance (Apple | | MFR | 0.20 |
| | Crunch #234-795) | | | |
| 8 | Putrescine | 333-93-7 | MFR | 0.80 |
| | dihydrochloride (1,4 | | | |
| | diaminobutane, | | | |
| | dihydrochloride) | | | |
| | | | | 100% |

The above formulation was prepared by adding, in a suitable stainless steel tank equipped with a lightening type propeller mixer, all ingredients one by one in the order they appear in Table 2 until the composition became clear and all ingredients have dissolved. The propeller mixer was set to medium to high speed and a nitrogen blanket was used throughout the entire manufacturing process (held at room temperature, i.e., about 21 degrees Celsius). Colored glass containers were used to store the finish product to reduce product contact with ambient light.

### Example 3

### Vitamin C 1% and Putrescine Deep Penetrating and Slow-Release Composition

TABLE 3
Exemplary 0.8% putrescine and 1% Vitamin C deep penetrating
and slow-release composition

| | | | | Amount |
|---|---|---|---|---|
| | Ingredients | CAS# | Grade | (% W/W) |
| 1 | Purified water | N.A. | USP | 19.0 |
| 2 | 3-O-ethyl-ascorbate | 86404-04-8 | USP | 1.0 |
| | (ET VC) | | | |
| 3 | Ethoxydiglycol | 111-90-0 | USP | 49.50 |
| | (Transcutol CG) | | | |
| 4 | Propylene Glycol | 57-55-6 | USP | 26.0 |
| 5 | Polysorbate 60 | 9005-67-8 | MFR | 1.30 |
| 6 | Betaine | 107-43-7 | MFR | 1.20 |
| 7 | Citrus Grandis | 90045-43-5, | MFR | 1.0 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.8 |
| | dihydrochloride (1,4 | | | |
| | diaminobutane, | | | |
| | dihydrochloride) | | | |
| | | | | 100% |

The above formulation was prepared by adding, in a suitable stainless steel tank equipped with a lightening type propeller mixer, all ingredients one by one in the order they appear in Table 3. At each step, the preparation was mixed for 15-10 minutes or until the ingredient added was fully dissolved and the preparation was clear. The propeller mixer was set to medium to high speed and a nitrogen blanket was used throughout the entire manufacturing process (held at room temperature, i.e., about 21 degrees Celsius). Colored glass containers were used to store the finish product to reduce product contact with ambient light.

### Example 4

### Vitamin C 5% and Putrescine Deep Penetrating and Slow-Release Composition

TABLE 4
Exemplary 0.8% putrescine and 5% Vitamin C deep penetrating
and slow-release composition

| | | | | Amount |
|---|---|---|---|---|
| | Ingredients | CAS# | Grade | (% W/W) |
| 1 | Purified water | N.A. | USP | 15.0 |
| 2 | 3-O-ethyl-ascorbate | 86404-04-8 | USP | 5.0 |
| | (ET VC) | | | |
| 3 | Ethoxydiglycol | 111-90-0 | USP | 49.50 |
| 4 | Propylene Glycol | 57-55-6 | USP | 26.0 |
| 5 | Polysorbate 60 | 9005-67-8 | MFR | 1.30 |
| 6 | Betaine | 107-43-7 | MFR | 1.20 |
| 7 | Citrus Grandis | 90045-43-5, | MFR | 1.0 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.8 |
| | dihydrochloride (1,4 | | | |
| | diaminobutane, | | | |
| | dihydrochloride) | | | |
| | | | | 100% |

The above composition was prepared by (i) starting with the required amount of purified water (10%); and adding (ii) 3.00% 3-O-ethyl-ascorbate or L-Ascorbic acid and mixing (at medium to high speed) for about 20 minutes or longer until fully dissolved; (iii) 24.75% ethoxydiglycol and mixing until fully dissolved; (iv) 2% of 3-O-ethyl-ascorbate and mixing (at medium to high speed) for about 20 minutes or longer until fully dissolved (v) 26.00% Propylene Glycol and mix for about 20 min.; (vi) 1.3% of Polysorbate 60 and 1.2% of Betaine and mix until fully dissolved; (vii) 1.00% antioxidant (Citrus grapefruit extract, Greentech™; (viii) 0.3% of fragrance (Apple Crunch™); and (ix) 0.8% of putrescine. Note: no heating was required. The manufacture of the above formulation was made in a stainless steel tank equipped with a lightening-type propeller mixer and colored glass containers were used to reduce product contact with ambient light. At each step, the preparation was mixed for about 20 minutes or until the ingredient added was fully dissolved and the preparation was homogeneous. The propeller mixer was set to medium to high speed (about 196 rpm for agitation and about 110 rpm for the homogenizer and a nitrogen blanket was used throughout the entire manufacturing process (held at room temperature, i.e., about 21 degrees Celsius). Note that at this concentration of Vitamin C (1%) no heating is required. Colored glass containers were used to store the finish product to reduce product contact with ambient light and increase stability of the product.

### Example 5

### Vitamin C 10% and Putrescine Deep Penetrating and Slow-Release Composition

TABLE 5
Exemplary 0.8% putrescine and 10% Vitamin C deep penetrating
and slow-release composition

| | | | | Amount |
|---|---|---|---|---|
| | Ingredients | CAS# | Grade | (% W/W) |
| 1 | Purified water | N.A. | USP | 10.0 |
| 2 | 3-O-ethyl-ascorbate | 86404-04-8 | USP | 10.0 |
| | (ET VC) | | | |
| 3 | Ethoxydiglycol | 111-90-0 | USP | 49.50 |
| 4 | Propylene Glycol | 57-55-6 | USP | 26.0 |
| 5 | Polysorbate 60 | 9005-67-8 | MFR | 1.30 |
| 6 | Betaine | 107-43-7 | MFR | 1.20 |
| 7 | Citrus Grandis | 90045-43-5, | MFR | 1.0 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.8 |
| | dihydrochloride (1,4 | | | |
| | diaminobutane, | | | |
| | dihydrochloride) | | | |
| | | | | 100% |

The above composition was prepared by adding and mixing the ingredients as follows in a stainless steel tank equipped with a lightening-type propeller mixer:

| Step | Manufacturing Procedure |
|---|---|
| 1 | Add Required amount of purified water (10.0%) to the tank. |
| 2 | Add 3.0% of Vit C (L-Ascorbic Acid USP and/or 3-O-ethyl-ascorbate), mix for about 20 |
| | minutes or longer until fully dissolved with an electric mixer. |
| 3 | Add about half (i.e., about 24.75%) of the required amount of ethoxydiglycol and mix for |
| | about 20 minutes or longer using an electric mixer until fully dissolved and clear. |
| 4 | Add 1.5% of Vit C (L-Ascorbic Acid USP and/or 3-O-ethyl-ascorbate), mix for about 20 |
| | minutes or longer until fully dissolved with an electric mixer. |
| 5 | Add other half (24.75%) of ethoxydiglycol and mix for about 20 minutes or longer using an |
| | electric mixer until fully dissolved and clear. |
| 6 | Add 3.5% L-Ascorbic Acid USP and/or 3-O-ethyl-ascorbate, mix for about 20 minutes or |
| | longer until fully dissolved with an electric mixer. |
| 7 | Add required amount of Propylene Glycol (27.0%) and mix for about 20 minutes or longer |
| | using an electric mixer until fully dissolved and clear. |
| 8 | Add required amount of Polysorbate 60 & Betaine and mix for about 15 minutes or longer |
| | using an electric mixer until fully dissolved and clear. |
| 9 | Add required amount of Grapefruit Extract (1.0%) and mix about 5 minutes. Begin heating to |
| | 40° C. |
| 10 | At approximately 32° C. add 2.0% L-Ascorbic Acid and/or 3-O-ethyl-ascorbate and continue |
| | to heat until 40° C. Ensure that the product does not exceed 40° C. While mixing, maintain at |
| | 40° C. until solution has become clear, free of any cloudiness with no crystals present. Turn |
| | off heat once 38° C. has been reached. Mixing time may vary with batch size. About 60 |
| | minutes of mixing was generally required to obtain a clear solution. |
| 11 | Sample the mixture using a beaker and check for undissolved crystals. If crystals are |
| | present, continue mixing. If no crystals are present, proceed to step 12. |
| 12 | Once fully dissolved and the solution is clear then add the Fragrance (0.20%). Remove from |
| | heat and allow to cool to 24° C.-28° C. Mix well for about 40 minutes. Mix until clear. |
| 13 | Sample top and bottom of container and check for undissolved crystals. If crystals are |
| | present, mix for about an additional 15 minutes. Repeat until solution is clear and fully |
| | dissolved. |
| 14 | Add putrescine HCl and mix until dissolved. |
| 15 | Weigh the batch and compensate for evaporation back to 100% with distilled water. Mix for |
| | about an additional 15 minutes. |
| 16 | Filter the batch using a 150 micron mesh filter (formulation should be clear and fully |
| | dissolved at this point. Filters cannot be relied on to remove undissolved crystals). |
| 17 | Apply nitrogen blanket to drum before sealing and preparing to fill the finished product. |

A nitrogen blanket was used throughout the entire manufacturing process. The formulation was prepared in a suitable stainless steel tank equipped with a lightening type propeller mixer and colored glass containers were used to prepare and store the final product to reduce product contact with ambient light. The pH of the final composition is between 2 and 4.

### Example 6

### Vitamin C 10% and Putrescine Slow-Release Composition

TABLE 6
Exemplary 0.4% putrescine and 10% Vitamin C
deep penetrating and slow-release composition

| | | | | Amount |
|---|---|---|---|---|
| | Ingredients | CAS# | Grade | (% W/W) |
| 1 | Purified water | N.A. | USP | 10.0 |
| 2 | L-Ascorbic Acid | 50-81-7 | USP | 10.0 |
| 3 | Ethoxydiglycol | 111-90-0 | USP | 51.3 |
| 4 | Propylene Glycol | 57-55-6 | USP | 27.10 |
| 5 | Citrus Grandis | 90045-43-5, | MFR | 1.0 |
| | Grapefruit Extract | 57-55-6 | | |
| | (Antioxydant; | | | |
| | Greentech) | | | |
| 6 | Apple Crunch #234- | | MFR | 0.20 |
| | 795 | | | |
| 7 | Putrescine | 333-93-7 | MFR | 0.4 |
| | dihydrochloride (1,4 | | | |
| | diaminobutane, | | | |
| | dihydrochloride) | | | |

100%

The above composition was prepared by adding and mixing the ingredients as follows in a stainless steel tank equipped with a lightening-type propeller mixer:

| Steps | Manufacturing Procedure |
|---|---|
| 1 | Add required amount of purified water (10.0%) to the tank. |
| 2 | Add 3.0% L-Ascorbic Acid USP, mix for about 20 minutes or longer until fully dissolved with an electric |
| | mixer. |
| 3 | Add 25.65% Ethoxydiglycol and mix for about 20 minutes or longer using an electric mixer until fully |
| | dissolved and clear. |
| 4 | Add 1.5% L-Ascorbic Acid USP, mix for about 20 minutes or longer until fully dissolved with an electric |
| | mixer. |
| 5 | Add 25.65% Ethoxydiglycol and mix for about 20 minutes or longer using an electric mixer until fully |
| | dissolved and clear. |
| 6 | Add 3.5% L-Ascorbic Acid USP, mix for about 20 minutes or longer until fully dissolved with an electric |
| | mixer. |
| 7 | Add required amount of Propylene Glycol (27.1%) and mix for about 20 minutes or longer using an |
| | electric mixer until fully dissolved and clear. |
| 8 | Add required amount of Grapefruit Extract (1.0%) and mix about 5 minutes. Begin heating to 40° C. |
| 9 | At approximately 32° C. add 2.0% L-Ascorbic Acid and continue to heat until 40° C. Ensure that the |
| | product does not exceed 40° C. While mixing, maintain at 40° C. until solution has become perfectly clear, |
| | free of any cloudiness and no crystals are present. Turn off heat once 38° C. has been reached. Mixing |
| | time may vary with batch size, however, about 60 minutes of mixing or more is generally required until |
| | solution is clear. |
| 10 | Sample the mixture using a beaker and check for undissolved crystals. If crystals are present, continue |
| | mixing. If no crystals are present, proceed to step 11. |
| 11 | Once fully dissolved and the solution is clear then add the Fragrance (0.20%). Remove from heat and |
| | allow to cool to 24° C.-28° C. Mix well for about 40 minutes. Mix until clear. |
| 12 | Sample top and bottom of container and check for undissolved crystals. If crystals are present, mix for |
| | about an additional 15 minutes. Repeat until solution is clear and fully dissolved. |
| 13 | Add 0.8% of Putrescine HCl and mix till completely dissolved about 45 min-1 hour. |
| 14 | Weigh the batch and compensate for evaporation back to 100% with distilled water. Mix for about an |
| | additional 15 minutes. |
| 15 | Filter the batch using a 150 microns mesh filter (solution should be clear and fully dissolved at this point, |
| | do not rely on filter to remove undissolved crystals). |
| 16 | Apply nitrogen blanket to drum before sealing and preparing to fill the finished product. |

A nitrogen blanket was used throughout the entire manufacturing process. The formulation was prepared in a suitable stainless steel tank equipped with a lightening type propeller mixer and colored glass containers were used to prepare and store the final product to reduce product contact with ambient light. The pH of the final composition is between 2 and 4.

### Example 7

### Vitamin C 10% and Putrescine 0.4% Deep Penetrating and Slow-Release Composition

TABLE 7
Exemplary 0.4% putrescine and 10% Vitamin C deep penetrating
and slow-release composition (Formula A)

| | | | | Amount |
|---|---|---|---|---|
| | Ingredients | CAS# | Grade | (% W/W) |
| 1 | Purified water | N.A. | USP | 20.0 |
| 2 | L-Ascorbic Acid | 50-81-7 | USP | 10.0 |
| 3 | Ethoxydiglycol | 111-90-0 | USP | 43.00 |
| 4 | Propylene Glycol | 57-55-6 | USP | 23.0 |
| 5 | Polysorbate 20 | 9005-67-8 | MFR | 1.30 |
| 6 | Betaine | 107-43-7 | MFR | 1.20 |
| 7 | Citrus Grandis | 90045-43-5, | MFR | 0.9 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.4 |
| | dihydrochloride (1,4 | | | |
| | diaminobutane, | | | |
| | dihydrochloride) | | | |
| | | | | 100% |

The above composition is prepared by adding and mixing the ingredients as follows in a stainless steel tank equipped with a lightening-type propeller mixer:

| Steps | Manufacturing Procedure |
|---|---|
| 1 | Add required amount of purified water (10.0%) to the tank. |
| 2 | Add 3.0% L-Ascorbic Acid USP, mix for about 20 minutes or longer until fully dissolved with an electric |
| | mixer. |
| 3 | Add 21.5% Ethoxydiglycol (i.e., about half) and mix for about 20 minutes or longer using an electric |
| | mixer until fully dissolved and clear. |
| 4 | Add 1.5% L-Ascorbic Acid USP, mix for about 20 minutes or longer until fully dissolved with an electric |
| | mixer. |
| 5 | Add 21.5% Ethoxydiglycol (i.e., about half) and mix for about 20 minutes or longer using an electric |
| | mixer until fully dissolved and clear. |
| 6 | Add 3.5% L-Ascorbic Acid USP, mix for 20 minutes or longer until fully dissolved with an electric mixer. |
| 7 | Add required amount of Propylene Glycol (23%) and mix for about 20 minutes or longer using an electric |
| | mixer until fully dissolved and clear. |
| 8 | Add required amount of Grapefruit Extract (0.9%) and mix about 5 minutes. Begin heating to 40° C. |
| 9 | Add required amount of betaine (1.2%) and mix until dissolution. |
| 10 | At approximately 32° C. add 2.0% L-Ascorbic Acid and continue to heat until 40° C. Ensure that the |
| | product does not exceed 40° C. While mixing, maintain at 40° C. until solution has become perfectly clear, |
| | free of any cloudiness and no crystals are present. Turn off heat once 38° C. has been reached. Mixing |
| | time may vary with batch size, however, about 60 minutes of mixing is generally required until solution is |
| | clear. |
| 11 | Sample the mixture using a beaker and check for undissolved crystals. If crystals are present, continue |
| | mixing. If no crystals are present, proceed to step 12. |
| 12 | Once fully dissolved and the solution is clear then add the Fragrance (0.20%). Remove from heat and |
| | allow to cool to 24° C.-28° C. Mix well for about 40 minutes or until until clear. |
| 13 | Sample top and bottom of container and check for undissolved crystals. If crystals are present, mix for |
| | about an additional 15 minutes. Repeat until solution is clear and fully dissolved. |
| 14 | Mix together 5% of distilled water and 0.4% of Putrescine HCl r. |
| 15 | Add the water putrescine mixture of step 14 to mixture obtained in step 13 and mix until completely |
| | dissolved. |
| 16 | Mix together 5% of distilled water and 1.3% of polysorbate 20 then add to the mixture of step 15. Mix |
| | until complete dissolution (about 20-45 min) |
| 15 | Weigh the batch and compensate for evaporation back to 100% with distilled water. Mix for about an |
| | additional 15 minutes. |
| 16 | Filter the batch using a 150 microns mesh filter (solution should be clear and fully dissolved at this point, |
| | do not rely on filter to remove undissolved crystals). |
| 17 | Apply nitrogen blanket to drum before sealing and preparing to fill the finished product. |

### Example 8

### Vitamin C 20% and Putrescine 0.4% Deep Penetrating and Slow-Release Composition

TABLE 8
Exemplary 0.4% putrescine and 20% Vitamin C deep penetrating
and slow-release composition (Formula B)

| | | | | Amount |
|---|---|---|---|---|
| | Ingredients | CAS# | Grade | (% W/W) |
| 1 | Purified water | N.A. | USP | 20 |
| 2 | 3-O-ethyl-ascorbate | 86404-04-8 | USP | 7.5 |
| | (ET VC) | | | |
| | L-Ascorbic Acid | | USP | 12.5 |
| 3 | Ethoxydiglycol | 111-90-0 | USP | 33.0 |
| 4 | Propylene Glycol | 57-55-6 | USP | 23.0 |
| 5 | Polysorbate 20 | 9005-67-8 | MFR | 1.30 |
| 6 | Betaine | 107-43-7 | MFR | 1.20 |
| 7 | Citrus Grandis | 90045-43-5, | MFR | 0.9 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.4 |
| | dihydrochloride (1,4 | | | |
| | diaminobutane, | | | |
| | dihydrochloride) | | | |

100%

The above composition was prepared by adding and mixing the ingredients as described in Example 7 in a stainless steel tank equipped with a lightening-type propeller mixer.

### Example 9

### Vitamin C 10% and Putrescine 0.8% Deep Penetrating and Slow-Release Composition

TABLE 9
Exemplary 0.4% putrescine and 20% Vitamin C deep penetrating
and slow-release composition (Formula C)

| | | | | Amount |
|---|---|---|---|---|
| | Ingredients | CAS# | Grade | (% W/W) |
| 1 | Purified water | N.A. | USP | 20 |
| 2 | L-Ascorbic Acid | | USP | 10 |
| 3 | Ethoxydiglycol | 111-90-0 | USP | 42.6 |
| 4 | Propylene Glycol | 57-55-6 | USP | 23.0 |
| 5 | Polysorbate 20 | 9005-67-8 | MFR | 1.30 |
| 6 | Betaine | 107-43-7 | MFR | 1.20 |
| 7 | Citrus Grandis | 90045-43-5, | MFR | 0.9 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.8 |
| | dihydrochloride (1,4 | | | |
| | diaminobutane, | | | |
| | dihydrochloride) | | | |
| | | | | 100% |

The above composition was prepared by adding and mixing the ingredients as described in Example 7 in a stainless steel tank equipped with a lightening-type propeller mixer.

### Example 10

### Vitamin C 20% and Putrescine 0.8% Deep Penetrating and Slow-Release Composition

TABLE 10
Exemplary 0.8% putrescine and 20% Vitamin C deep penetrating
and slow-release composition (Formula D)

| | | | | Amount |
|---|---|---|---|---|
| | Ingredients | CAS# | Grade | (% W/W) |
| 1 | Purified water | N.A. | USP | 20.00 |
| 2 | 3-O-ethyl-ascorbate | 86404-04-8 | USP | 7.5 |
| | (ET VC) | | | |
| | L-Ascorbic Acid | | USP | 12.5 |
| 3 | Ethoxydiglycol | 111-90-0 | USP | 33.0 |
| 4 | Propylene Glycol | 57-55-6 | USP | 22.6 |
| 5 | Polysorbate 20 | 9005-67-8 | MFR | 1.30 |
| 6 | Betaine | 107-43-7 | MFR | 1.20 |
| 7 | Citrus Grandis | 90045-43-5, | MFR | 0.9 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.8 |
| | dihydrochloride (1,4 | | | |
| | diaminobutane, | | | |
| | dihydrochloride) | | | |
| | | | | 100% |

The above composition was prepared by adding and mixing the ingredients as described in Example 7 in a stainless steel tank equipped with a lightening-type propeller mixer.

### Example 11

### Vitamin C 10% and Putrescine 0.4% and Ethoxydiglycol 2.5% Deep Penetrating and Slow-Release

TABLE 11
Exemplary 0.4% putrescine and 10% Vitamin C deep penetrating
and slow-release composition (Formula: E)

| | | | | Amount |
|---|---|---|---|---|
| | Ingredients | CAS# | Grade | (% W/W) |
| 1 | Purified water | N.A. | USP | 20.00 |
| 2 | L-Ascorbic Acid | 50-81-7 | USP | 10.0 |
| 3 | Ethoxydiglycol | 111-90-0 | USP | 2.5 |
| 4 | Propylene Glycol | 57-55-6 | USP | 31.9 |
| 5 | Butylene Glycol | 107-88-0 | USP | 22.0 |
| 6 | Pentylene Glycol | 5343-92-0 | USP | 22.0 |
| 7 | Citrus Grandis | 90045-43-5, | MFR | 1.0 |
| | Grapefruit Extract | 57-55-6 | | |
| | (Antioxydant; | | | |
| | Greentech) | | | |
| 8 | Apple Crunch #234- | | MFR | 0.20 |
| | 795 | | | |
| 9 | Putrescine | 333-93-7 | MFR | 0.4 |
| | dihydrochloride (1,4 | | | |
| | diaminobutane, | | | |
| | dihydrochloride) | | | |

100%

The above composition was prepared by adding and mixing the ingredients as follows in a stainless steel tank equipped with a lightening-type propeller mixer:

| Steps | Manufacturing Procedure |
|---|---|
| 1 | Add required amount of purified water (10.0%) to the tank. |
| 2 | Add 3.0% L-Ascorbic Acid USP, mix for about 20 minutes or longer until fully dissolved with an electric |
| | mixer. |
| 3 | Add 2.5% Ethoxydiglycol, 22% Butylene Glycol and mix for about 20 minutes or longer using an electric |
| | mixer until fully dissolved and clear. |
| 4 | Add 1.5% L-Ascorbic Acid USP, mix for 20 minutes or longer until fully dissolved with an electric mixer. |
| 5 | Add 22% Pentylene Glycol and mix for about 20 minutes or longer using an electric mixer until fully |
| | dissolved and clear. |
| 6 | Add 3.5% L-Ascorbic Acid USP, mix for about 20 minutes or longer until fully dissolved with an electric |
| | mixer. |
| 7 | Add required amount of Propylene Glycol (31.9%) and mix for about 20 minutes or longer using an |
| | electric mixer until fully dissolved and clear. |
| 8 | Add required amount of Grapefruit Extract (1.0%) and mix for about 5 minutes. Begin heating to 40° C. |
| 9 | At approximately 32° C. add 2.0% L-Ascorbic Acid and continue to heat until 40° C. Ensure that the |
| | product does not exceed 40° C. While mixing, maintain at 40° C. until solution has become perfectly clear, |
| | free of any cloudiness and no crystals are present. Turn off heat once 38° C. has been reached. Mixing |
| | time may vary with batch size, however, generally 60 minutes or more of mixing is required until solution |
| | is clear. |
| 10 | Sample the mixture using a beaker and check for undissolved crystals. If crystals are present, continue |
| | mixing. If no crystals are present, proceed to step 11. |
| 11 | Once fully dissolved and the solution is clear then add the Fragrance (0.20%). Remove from heat and |
| | allow to cool to 24° C.-28° C. Mix well for about 40 minutes or until until clear. |
| 12 | Sample top and bottom of container and check for undissolved crystals. If crystals are present, mix for |
| | about an additional 15 minutes. Repeat until solution is clear and fully dissolved. |
| 13 | Add 0.4% of 1,4-Diaminobutane Dihydrochloride and mix until completely dissolved i.e., about 45 min-1 |
| | hour. |
| 14 | Weigh the batch and compensate for evaporation back to 100% with purified water. Mix for about 15 |
| | minutes. |
| 15 | Filter the batch using a 150 Micron mesh filter (Solution must be clear and fully dissolved at this point, do |
| | not rely on filter to remove undissolved crystals). |
| 16 | Apply Nitrogen Blanket to drum before sealing and preparing to fill the finished product. |

### Example 12

### Vitamin C 10% and Putrescine 0.4% and Ethoxydiglycol 2.5% Deep Penetrating and Slow-Release

TABLE 12
Exemplary 0.4% putrescine and 10% Vitamin C deep penetrating
and slow-release composition (Formula: F)

| | | | | Amount |
|---|---|---|---|---|
| | Ingredients | CAS# | Grade | (% W/W) |
| 1 | Purified water | N.A. | USP | 20.00 |
| 2 | L-Ascorbic Acid | 50-81-7 | USP | 5.0 |
| 3 | 3-O-Ethyl Ascorbic | 86404-04-8 | MFR | 5.0 |
| | Acid | | | |
| 4 | Ethoxydiglycol | 111-90-0 | USP | 2.5 |
| 5 | Propylene Glycol | 57-55-6 | USP | 31.9 |
| 6 | Butylene Glycol | 107-88-0 | USP | 22.0 |
| 7 | Pentylene Glycol | 5343-92-0 | USP | 22.0 |
| 8 | Citrus Grandis | 90045-43-5, | MFR | 1.0 |
| | Grapefruit Extract | 57-55-6 | | |
| | (Antioxydant; | | | |
| | Greentech) | | | |
| 9 | Apple Crunch #234- | | MFR | 0.20 |
| | 795 | | | |
| 10 | Putrescine | 333-93-7 | MFR | 0.4 |
| | dihydrochloride (1,4 | | | |
| | diaminobutane, | | | |
| | dihydrochloride) | | | |
| | | | | 100% |

The above composition was prepared by adding and mixing the ingredients in a stainless steel tank equipped with a lightening-type propeller mixer:

| Steps | Manufacturing Procedure |
|---|---|
| 1 | Add required amount of purified water (10.0%) to the tank. |
| 2 | Add 3.0% L-Ascorbic Acid USP, mix for about 20 minutes or longer until fully dissolved with an electric |
| | mixer. |
| 3 | Add 2.5% Ethoxydiglycol, 22% Butylene Glycol and mix for about 20 minutes or longer using an electric |
| | mixer until fully dissolved and clear. |
| 4 | Add 2.0% L-Ascorbic Acid USP, mix for about 20 minutes or longer until fully dissolved with an electric |
| | mixer. |
| 5 | Add 22% Pentylene Glycol and mix for about 20 minutes or longer using an electric mixer until fully |
| | dissolved and clear. |
| 6 | Add 3.0% 3-O-Ethyl Ascorbic Acid, mix for about 20 minutes or longer until fully dissolved with an |
| | electric mixer. |
| 7 | Add required amount of Propylene Glycol (27.1%) and mix for about 20 minutes or longer using an |
| | electric mixer until fully dissolved and clear. |
| 8 | Add required amount of Grapefruit Extract (1.0%) and mix 5 minutes. Begin heating to 40° C. |
| 9 | At approximately 32° C. add 2.0% 3-O-Ethyl Ascorbic Acid and continue to heat until 40° C. Ensure that |
| | the product does not exceed 40° C. While mixing, maintain at 40° C. until solution has become perfectly |
| | clear, free of any cloudiness and no crystals are present. Turn off heat once 38° C. has been reached. |
| | Mixing time may vary with batch size, however, about 60 minutes of mixing or more is generally required |
| | until solution is clear. |
| 10 | Sample the mixture using a beaker and check for undissolved crystals. If crystals are present, continue |
| | mixing. If no crystals are present, proceed to step 11. |
| 11 | Once fully dissolved and the solution is clear then add the Fragrance (0.20%). Remove from heat and |
| | allow to cool to 24° C.-28° C. Mix well for about 40 minutes. Mix until clear. |
| 12 | Sample top and bottom of container and check for undissolved crystals. If crystals are present, mix for |
| | about an additional 15 minutes. Repeat until solution is clear and fully dissolved. |
| 13 | Add 0.4% of 1,4-Diaminobutane Dihydrochloride and mix till completely dissolved about 45 min-1 hour. |
| 14 | Weigh the batch and compensate for evaporation back to 100% with purified water. Mix for about an |
| | additional 15 minutes. |
| 15 | Filter the batch using a 150 Micron mesh filter (Solution must be clear and fully dissolved at this point, do |
| | not rely on filter to remove undissolved crystals). |
| 16 | Apply Nitrogen Blanket to drum before sealing and preparing to fill the finished product. |

### Example 13

### Vitamin C 10% and Putrescine 0.4% and Ethoxydiglycol Degree 2.5% Deep Penetrating and Slow-Release

TABLE 13
Exemplary 0.4% putrescine and 10% Vitamin C deep penetrating
and slow-release composition (Formula: G)

| | | | | Amount |
|---|---|---|---|---|
| | Ingredients | CAS# | Grade | (% W/W) |
| 1 | Purified water | N.A. | USP | 20.00 |
| 2 | L-Ascorbic Acid | 50-81-7 | USP | 3.0 |
| 3 | 3-O-Ethyl Ascorbic | 86404-04-8 | MFR | 7.0 |
| | Acid | | | |
| 4 | Ethoxydiglycol | 111-90-0 | USP | 2.5 |
| 5 | Propylene Glycol | 57-55-6 | USP | 31.9 |
| 6 | Butylene Glycol | 107-88-0 | USP | 22.0 |
| 7 | Pentylene Glycol | 5343-92-0 | USP | 22.0 |
| 8 | Citrus Grandis | 90045-43-5, | MFR | 1.0 |
| | Grapefruit Extract | 57-55-6 | | |
| | (Antioxydant; | | | |
| | Greentech) | | | |
| 9 | Apple Crunch #234- | | MFR | 0.20 |
| | 795 | | | |
| 10 | Putrescine | 333-93-7 | MFR | 0.4 |
| | dihydrochloride (1,4 | | | |
| | diaminobutane, | | | |
| | dihydrochloride) | | | |

100%

The above composition was prepared by adding and mixing the ingredients in a stainless steel tank equipped with a lightening-type propeller mixer:

| Steps | Manufacturing Procedure |
|---|---|
| 1 | Add required amount of purified water (10.0%) to the tank. |
| 2 | Add 3.0% L-Ascorbic Acid USP, mix for about 20 minutes or longer until fully dissolved with an electric |
| | mixer. |
| 3 | Add 2.5% Ethoxydiglycol, 22% Butylene Glycol and mix for about 20 minutes or longer using an electric |
| | mixer until fully dissolved and clear. |
| 4 | Add 2.0% 3-O-Ethyl Ascorbic Acid, mix for about 20 minutes or longer until fully dissolved with an |
| | electric mixer. |
| 5 | Add 22% Pentylene Glycol and mix for about 20 minutes or longer using an electric mixer until fully |
| | dissolved and clear. |
| 6 | Add 3.0% 3-O-Ethyl Ascorbic Acid, mix for about 20 minutes or longer until fully dissolved with an |
| | electric mixer. |
| 7 | Add required amount of Propylene Glycol (27.1%) and mix for about 20 minutes or longer using an |
| | electric mixer until fully dissolved and clear. |
| 8 | Add required amount of Grapefruit Extract (1.0%) and mix for about 5 minutes. Begin heating to 40° C. |
| 9 | At approximately 32° C. add 2.0% 3-O-Ethyl Ascorbic Acid and continue to heat until 40° C. Ensure that |
| | the product does not exceed 40° C. While mixing, maintain at 40° C. until solution has become perfectly |
| | clear, free of any cloudiness and no crystals are present. Turn off heat once 38° C. has been reached. |
| | Mixing time may vary with batch size, however, about 60 minutes of mixing or more is generally required |
| | until solution is clear. |
| 10 | Sample the mixture using a beaker and check for undissolved crystals. If crystals are present, continue |
| | mixing. If no crystals are present, proceed to step 11. |
| 11 | Once fully dissolved and the solution is clear then add the Fragrance (0.20%). Remove from heat and |
| | allow to cool to 24° C.-28° C. Mix well for about 40 minutes. Mix until clear. |
| 12 | Sample top and bottom of container and check for undissolved crystals. If crystals are present, mix for |
| | about an additional 15 minutes. Repeat until solution is clear and fully dissolved. |
| 13 | Add 0.4% of 1,4-Diaminobutane Dihydrochloride and mix till completely dissolved about 45 min-1 hour. |
| 14 | Weigh the batch and compensate for evaporation back to 100% with purified water. Mix for about |
| | additional 15 minutes. |
| 15 | Filter the batch using a 150 Micron mesh filter (Solution must be clear and fully dissolved at this point, do |
| | not rely on filter to remove undissolved crystals). |
| 16 | Apply Nitrogen Blanket to drum before sealing and preparing to fill the finished product. |

### Example 14

### Vitamin C Formulation Allows for Efficient Skin Penetration

Topical products can differ in how much a given active ingredient actually penetrates and remains within the skin. How deep active ingredients penetrate is important because it affects their biological effects and thus, how effective a given composition will be. For example, L-ascorbic acid can only exert its beneficial effects if it physically gets inside the fibroblasts and other cells located within the various layers of the skin. Compositions of the present invention contain a transdermal system which is used to maximize skin penetration of active ingredients such as Vitamin C and putrescine.

A study was completed at the University of California, Irvine, comparing the ability of a composition of the present invention comprising 10% of Vitamin C and a Vitamin C serum from Skinceuticals® (also comprising 10% of Vitamin C) to penetrate the stratum corneum and deliver Vitamin C to the epidermis and dermis. Twenty-four small samples of cadaver skin, less than 2 cm² each, were used in testing for each product. Each skin sample was sealed in contact with, and directly above, a small individual reservoir containing approximately 10 mL of saline solution. The dermis was placed directly into contact with the saline solution, while the stratum corneum remained exposed to the air. Very small but carefully defined amount of radioactively-labeled Vitamin C were spiked into the two products before they were applied to the skin samples. This radiolabeled Vitamin C was then used as a tab to quantify the overall amount of Vitamin C to be found in the reservoir and within the skin samples, once the test got underway. To begin the test, 30 mg of each product were gently spread on top of each skin sample (stratum corneum). Then at 0, 1, 6, 12 and 24 hours after this single skin application, radiolabeled Vitamin C was measured in the saline reservoir, enabling the total amount of Vitamin C that penetrated through the skin and into the reservoir to be calculated for each time point. At the end of the study (24 hours), the skin samples were removed from test apparatus, the three skin layers carefully separated, and the amount of Vitamin C within each layer was measured. As expected, the amount of Vitamin C in the reservoir increased at each time point for both products, indicating that the Vitamin C was penetration the skin over time. There was no statistically significant difference between the two products in terms of how much Vitamin C moved from one side of the skin sample out the other (see “Reservoir” in Table 8 below). However when the amount of Vitamin C remaining in the stratum corneum, epidermis and dermis was compared for the two products at the end of the study period (24 hours), the composition of the present invention was shown to provide significantly increased levels of Vitamin C within all three skin compartments (see Table 8, below).

TABLE 14
Serum with 10% Vitamin C formulation (Serum 10)

| | | | | Amount |
|---|---|---|---|---|
| Ingredients | CAS# | TM | Grade | (% W/W) |
| Demineralized water | N.A. | | USP | 10. |
| L-Ascorbic acid | 50-81-7 | Hoffman | USP | 10.0 |
| | | Laroche | | |
| Ethoxydiglycol | 111-90-0 | Trivalin SF ™ | USP | 51.8 |
| Citrus Grandis | 90045-43-5, | Greentech ™ | MFR | 1.0 |
| Grapefruit Extract | 57-55-6 | | | |
| (Antioxydant) | | | | |
| Fragrance | | | MFR | 0.2 |
| Propylene Glycol | 57-55-6 | | USP | 27.0 |

100%

The above composition was prepared as described in Example 6 but without the addition of putrescine (1,4-Diaminobutane).

TABLE 15
Localization of Vitamin C at 24 hours (% of initial dose)

| | | | S. | | |
|---|---|---|---|---|---|
| | Dermis | Epidermis | Corneum | Reservoir | Other* |
| Slow-release | 0.9 | 3.6 | 1.1 | 7.6 | 86.8 |
| formulation | | | | | |
| comprising 10% | | | | | |
| L-ascorbic acid | | | | | |
| Skinceuticals | 0.3 | 0.8 | 0.4 | 5.1 | 93.4 |
| P Value** | <0.001 | <0.001 | <0.006 | 0.4 | — |

*Includes remainder of Vitamin C recovered from s. corneum surface, in washes on applicator tips, gauze, etc.
**P values less than 0.05 are considered to indicate a true difference in results, i.e. not likely to have occurred by chance.

By the end of the study period, 5.5% of the initial dose of Serum 10 was found in one of the three skin compartments. In contrast, only 1.5% of the initial dose of Skinceuticals. Vitamin C could be found in these three skin layers. The remainder of the Vitamin C in these tests was recovered elsewhere. Vitamin C is known to increase collagen production and in other ways help reverse the impact of sun damage to the skin. Several aspects of topical Vitamin C product formulation help determine the likelihood of therapeutic results and patient satisfaction. These include the use of the L-ascorbic acid form of Vitamin C, a low pH vehicle for shelf stability, and the use of an effective skin penetrating agent such as ethoxydiglycol. The UCI study showed that, when compared to the Skinceuticals Vitamin C product, Serum 10 provided 4 times the amount of Vitamin C to the epidermis and dermis, the two key skin compartments where Vitamin C can play a role in reversing the effects of aging and skin damage, including sun damage. This difference in the delivery of Vitamin C is statistically significant, i.e. carries a 95% probability that it is not just a chance finding. Based on the careful measurements documented in this study, Serum 10 was superior to Skinceuticals in delivering Vitamin C to the target tissues.

### Example 15

### Stability of Compositions of the Present Invention

Preliminary stability tests of compositions of the present invention comprising putrescine (from 0.4% or 0.8%) and optionally 1%, 5% or 10% Vitamin C (3-O-3thyl Ascorbic acid) show that the compositions are stable. Indeed, no significant change in color, odor, pH and texture (including absence of multiple phases, and precipitate) were observed after about 12 months. Further stability tests are underway and are conducted as follows.

TABLE 15
Stability program design for putrescine and/or vitamin C slow
release formulations
25° C./+/−2 to 75% RH

| | Time points (months) | | | | | | | | | | |
|---|---|---|---|---|---|---|---|---|---|---|---|
| | 0 | 3 | 6 | 9 | 12 | 18 | 24 | 30 | 36 | 42 | 48 |
| Organoleptic | C | C | C | C | C | C | C | C | C | C | C |
| pH USP <791>¹ | C | C | C | C | C | C | C | C | C | C | C |
| Viscosity USP | C | C | C | C | C | C | C | C | C | C | C |
| <912>² | | | | | | | | | | | |
| Vitamin C (HPLC)³ | C | C | C | C | C | C | C | C | C | C | C |
| Putrescine (HPLC)⁴ | C | C | C | C | C | C | C | C | C | C | C |
| Microbiology | C | C | C | C | C | C | C | C | C | C | C |
| USP<62>⁵ AND | | | | | | | | | | | |
| <62>⁶ | | | | | | | | | | | |

C = Anticipate Results should be conform to all testing specifications
¹The pH value similar to the initial one over time ±10%
²The Viscosity similar to the initial one over time ±10%: will be performed every 6 months only.
³The Vitamin C ±10% of the initial concentration of the formula, is present
⁴The Putrescine ±10% of the initial concentration of the formula
⁵USP<61> = Total Aerobic Count <100 cfu/g & Yeasts and Moulds <100 cfu/g
⁶USP<62> = S. aureus, P. aeruginosa, E. coli, Salmonella sp. all absent

TABLE 17
Stability of Serum Formula described in Example 9 (10% L-Ascorbic Acid, 0.4% Putrescine)

| | | | Finished Product Testing Specification | | | |
|---|---|---|---|---|---|---|
| | | | VS-44-02 | VS-44-02 | VS-44-02 | VS-44-04 |
| | | | Laboratory | | | |
| | | | MS Pharma/Alpharmco | | | |
| | | Method or | Initial | | | MS Pharma/EnvironeX |
| Test | Specification | Reference | Testing | 3 Mth | 6 Mth | 9 Mth |
| Appearance | To report | Organoleptic | Clear | Clear pale yellow | Clear pale | Clear pale orange liquid, |
| | (Clear colorless to pale | | colorless | liquid | orange liquid | free from foreign material |
| | orange | | liquid | | free from | |
| | Liquid, free form foreign | | | | foreign material | |
| | material) | | | | | |
| pH | 2.0-4.0 | USP <791> | 2.7 | 2.7 | 2.8 | 2.7 |
| (10% solution) | | | | | | |
| Viscosity | to report | USP <912> | 52 | NT | 59.4 | NT |
| Assay | to report | QIN-10-205 | 0.4 | 0.3 | 0.4 | 0.3 |
| 1,4- | (target: 0.4%) | | | | | |
| Diaminobutane | | | | | | |
| Dihydrochloride | | | | | | |
| (%) | | | | | | |
| Assay | to report | QIN-10-145 | 9.5 | 10.0 | 9.8 | 10.0 |
| Ascorbic Acid | (target: 10%) | | | | | |
| (%) | | | | | | |
| Microbiology | Total Aerobic Count ≤100 cfu/g | USP <61> | <10 cfu/g | <10 cfu/g | <10 cfu/g | <10 cfu/g |
| | Yeast & Molds ≤100 cfu/g | USP<61> | <10 cfu/g | <10 cfu/g | <10 cfu/g | <10 cfu/g |
| | E. coli (ABSENT) | USP<62> | Not detected | Not detected | Not detected | Not detected |
| | S. aureus (ABSENT) | USP<62> | Not detected | Not detected | Not detected | Not detected |
| | P. aeruginosa (ABSENT) | USP<62> | Not detected | Not detected | Not detected | Not detected |
| | Salmonella (ABSENT) | USP<62> | Not detected | Not detected | Not detected | Not detected |

NT: Not tested
Manufacturing date: December 2016.
Packaging: 30 ml flint glass bottle sprayed with PMS 877.

TABLE 18
Stability of slow release and deep penetrating Formula A described in Example 7
(10% 3-O-Ethyl Ascorbic Acid, 0.4% Putrescine)

| | | | Finished Product Testing Specification | | | |
|---|---|---|---|---|---|---|
| | | | VS-44-02 | RD-11-00 | RD-11-11 | Rd-11-00 |
| | | | Laboratory | | | |
| | | | MS Pharma/ | | | |
| | | | Alpharmco | MS Pharma/ | MS Pharma/ | MS Pharma/ |
| | | Method or | Initial | Alpharmco | EnvironeX | EnvironeX |
| Test | Specification | Reference | Testing | 3 Mth | 6 Mth | 9 Mth |
| Appearance | Clear colorless to pale | Organoleptic | C | C | C | C |
| | orange | | | | | |
| | Liquid, free form foreign | | | | | |
| | material | | | | | |
| pH | 2.0-4.0 | USP <791> | 2.6 | 2.8 | 2.8 | 2.9 |
| (10% solution) | | | | | | |
| Assay | to report | QIN-10-205 | 0.5 | 0.3 | 0.4 | 0.4 |
| 1,4- | (target: 0.4%) | | | | | |
| Diaminobutane | | | | | | |
| Dihydrochloride | | | | | | |
| (%) | | | | | | |
| Assay | to report | QIN-10-145 | 9.9 | 10 | 10 | 10 |
| Ascorbic Acid | (target: 10%) | | | | | |
| (%) | | | | | | |
| Microbiology | Total Aerobic Count ≤100 cfu/g | USP <61> | <10 cfu/g | <10 cfu/g | <10 cfu/g | <10 cfu/g |
| | Yeast & Molds ≤100 cfu/g | USP<61> | <10 cfu/g | <10 cfu/g | <10 cfu/g | <10 cfu/g |
| | E. coli (ABSENT) | USP<62> | not detected | not detected | not detected | not detected |
| | S. aureus (ABSENT) | USP<62> | not detected | not detected | not detected | not detected |
| | P. aeruginosa (ABSENT) | USP<62> | not detected | not detected | not detected | not detected |
| | Salmonella (ABSENT) | USP<62> | not detected | not detected | not detected | not detected |

NT: Not tested;
C: Conforms
Manufacturing date: January 2017,
Packaging: 30 ml flint glass bottle sprayed with PMS 877.

TABLE 19
Stability of slow release and deep penetrating Formula B described in Example 8
(12.5% L-Ascorbic Acid, 7.5% 3-O-Ethyl Ascorbic Acid, 0.4% Putrescine)

| | | | Finished Product Testing Specification | | | |
|---|---|---|---|---|---|---|
| | | | RD-10-00 | RD-10-00 | RD-10-00 | RD-10-01 |
| | | | Laboratory | | | |
| | | | MS Pharma/ | | | |
| | | | Alpharmco | MS Pharma/ | MS Pharma/ | MS Pharma/ |
| | | Method or | Initial | Alpharmco | Alpharmco | EnvironeX |
| Test | Specification | Reference | Testing | 3 Mth | 6 Mth | 9 Mth |
| Appearance | Clear colorless to pale | Organoleptic | C | C | C | C |
| | orange | | | | | |
| | Liquid, free form foreign | | | | | |
| | material | | | | | |
| pH | 2.0-4.0 | USP <791> | 2.6 | 2.7 | 2.8 | 2.8 |
| (10% solution) | | | | | | |
| Assay | to report | QIN-10-205 | 0.5 | 0.4 | 0.4 | 0.3 |
| 1,4- | (target: 0.4%) | | | | | |
| Diaminobutane | | | | | | |
| Dihydrochloride | | | | | | |
| (%) | | | | | | |
| Assay | to report | QIN-10-145 | 12.4 | 12.2 | 12.5 | 12.3 |
| Ascorbic Acid | (target: 10%) | | | | | |
| (%) | | | | | | |
| Assay 3-O Ethyl | To report | QIN-10-145 | 7.6 | 7.6 | 7.6 | 7.6 |
| Ascoric Acid | (target 7.5%) | | | | | |
| Microbiology | Total Aerobic Count ≤100 cfu/g | USP <61> | <10 cfu/g | <10 cfu/g | <10 cfu/g | <10 cfu/g |
| | Yeast & Molds ≤100 cfu/g | USP<61> | <10 cfu/g | <10 cfu/g | <10 cfu/g | <10 cfu/g |
| | E. coli (ABSENT) | USP<62> | not detected | not detected | not detected | not detected |
| | S. aureus (ABSENT) | USP<62> | not detected | not detected | not detected | not detected |
| | P. aeruginosa (ABSENT) | USP<62> | not detected | not detected | not detected | not detected |
| | Salmonella (ABSENT) | USP<62> | not detected | not detected | not detected | not detected |

NT: Not tested;
C: Conforms
Manufacturing date: January 2017.
Packaging: 30 ml flint glass bottle sprayed with PMS 877.

TABLE 20
Stability of slow release and deep penetrating Formula C described in Example 9
(10% 3-O-Ethyl Ascorbic Acid, 0.8% Putrescine)

| | | | Finished Product Testing Specification | | | |
|---|---|---|---|---|---|---|
| | | | VS-44-02 | RD-11-00 | RD-11-11 | Rd-11-00 |
| | | | Laboratory | | | |
| | | | MS Pharma/ | | | |
| | | | Alpharmco | MS Pharma/ | MS Pharma/ | MS Pharma/ |
| | | Method or | Initial | Alpharmco | Alpharmco | EnvironeX |
| Test | Specification | Reference | Testing | 3 Mth | 6 Mth | 9 Mth |
| Appearance | Clear colorless to pale | Organoleptic | C | C | C | C |
| | orange | | | | | |
| | Liquid, free form foreign | | | | | |
| | material | | | | | |
| pH | 2.0-4.0 | USP <791> | 2.7 | 2.8 | 2.8 | 2.8 |
| (10% solution) | | | | | | |
| Assay | to report | QIN-10-205 | 0.9 | 0.7 | 0.8 | 0.7 |
| 1,4- | (target: 0.4%) | | | | | |
| Diaminobutane | | | | | | |
| Dihydrochloride | | | | | | |
| (%) | | | | | | |
| Assay | to report | QIN-10-145 | 10 | 10 | 10 | 10 |
| Ascorbic Acid | (target: 10%) | | | | | |
| (%) | | | | | | |
| Microbiology | Total Aerobic Count ≤100 cfu/g | USP <61> | <10 cfu/g | <10 cfu/g | <10 cfu/g | <10 cfu/g |
| | Yeast & Molds ≤100 cfu/g | USP<61> | <10 cfu/g | <10 cfu/g | <10 cfu/g | <10 cfu/g |
| | E. coli (ABSENT) | USP<62> | not detected | not detected | not detected | not detected |
| | S. aureus (ABSENT) | USP<62> | not detected | not detected | not detected | not detected |
| | P. aeruginosa (ABSENT) | USP<62> | not detected | not detected | not detected | not detected |
| | Salmonella (ABSENT) | USP<62> | not detected | not detected | not detected | not detected |

NT: Not tested;
C: Conforms
Manufacturing date: January 2017.
Packaging: 30 ml flint glass bottle sprayed with PMS 877.

TABLE 21
Stability of slow release and deep penetrating Formula D described in Example 10
(12.5% L-Ascorbic Acid, 7.5% 3-O-Ethyl Ascorbic Acid, 0.8% Putrescine)

| | | | Finished Product Testing Specification | | | |
|---|---|---|---|---|---|---|
| | | | RD-10-00 | RD-10-00 | RD-10-00 | RD-10-01 |
| | | | Laboratory | | | |
| | | | MS Pharma/ | | | |
| | | | Alpharmco | MS Pharma/ | MS Pharma/ | MS Pharma/ |
| | | Method or | Initial | Alpharmco | Alpharmco | EnvironeX |
| Test | Specification | Reference | Testing | 3 Mth | 6 Mth | 9 Mth |
| Appearance | Clear colorless to pale | Organoleptic | C | C | C | C |
| | orange | | | | | |
| | Liquid, free form foreign | | | | | |
| | material | | | | | |
| pH | 2.0-4.0 | USP <791> | 2.7 | 2.7 | 2.7 | 2.8 |
| (10% solution) | | | | | | |
| Viscosity | to report | USP <912> | NT | NT | NT | NT |
| Assay | to report | QIN-10-205 | 0.9 | 0.7 | 0.7 | 0.7 |
| 1,4- | (target: 0.8%) | | | | | |
| Diaminobutane | | | | | | |
| Dihydrochloride | | | | | | |
| (%) | | | | | | |
| Assay | to report | QIN-10-145 | 12.4 | 12.3 | 12.5 | 12.2 |
| Ascorbic Acid | (target: 10%) | | | | | |
| (%) | | | | | | |
| Assay 3-O | To report | QIN-10-145 | 7.7 | 7.6 | 7.6 | 7.6 |
| Ethyl | (target 7.5%) | | | | | |
| Ascoric Acid | | | | | | |
| Microbiology | Total Aerobic Count ≤100 cfu/g | USP <61> | <10 cfu/g | <10 cfu/g | <10 cfu/g | <10 cfu/g |
| | Yeast & Molds ≤100 cfu/g | USP<61> | <10 cfu/g | <10 cfu/g | <10 cfu/g | <10 cfu/g |
| | E. coli (ABSENT) | USP<62> | not detected | not detected | not detected | not detected |
| | S. aureus (ABSENT) | USP<62> | not detected | not detected | not detected | not detected |
| | P. aeruginosa (ABSENT) | USP<62> | not detected | not detected | not detected | not detected |
| | Salmonella (ABSENT) | USP<62> | not detected | not detected | not detected | not detected |

NT: Not tested;
C: Conforms
Manufacturing date: January 2017.
Packaging: 30 ml flint glass bottle sprayed with PMS 877

### Example 16

### Putrescine Increases Skin Thickness

The effects a basolaterally applied 0.1% putrescine (1,4-Diaminobutane dihydrochloride in culture medium (EFT-400)) on epidermal thickness in the EpiDermFT™ (model, EFT-400, MatTek Corporation, Ashland, Mass.) human in vitro tissue model was assessed. EFT-400 tissues were treated with test compound every 48 hours for 4, 7 and 10 days.

Epidermal thickness was assessed by measuring 10 random regions of the Hematoxylin and Eosin (H&E) stained tissue sections using OlyVIA™ software. Measurements are reported in Table 18 below and representative H&E stainings are shown in FIG. 1. Exemplary Photographs of epidermal thickness of the untreated tissues was on an average 80.264 μm while tissues treated with 0.1% putrescine was found to be 117.561 μm (an average increase of over 46%).

At day 4, tissues treated with 0.1% Putrescine, contained basal and suprabasal layers with viable, nucleated cells. Basal cell nuclei appeared normal. No vacuolization within the epidermis was evident. Morphology was consistent with healthy tissue.

Also, EFT-400 tissues histological cross-sections treated or not with 0.1% putrescine were immunostained for Cytokeratin 10 (GREEN) and desmoplakin-1 (BLUE) to assess the effect of treatment on tissue thickness. After 4 days, epidermal tissues treated with 0.1% Putrescine visually appear thicker than the untreated control tissues (see FIG. 2).

TABLE 22
Epidermal thickness measurements following putrescine treatment

| | Untreated | | | 0.1% Test Article | | | |
|---|---|---|---|---|---|---|---|
| | Tissue 1 | Tissue 2 | Tissue 3 | Tissue 1 | Tissue 2 | Tissue 3 | |
| | Epidermis | Epidermis | Epidermis | Epidermis | Epidermis | Epidermis | |
| | Tickness | Tickness | Tickness | Tickness | Tickness | Tickness | |
| | [μm] | [μm] | [μm] | [μm] | [μm] | [μm] | |
| 1 | 78.9 | 72.81 | 83.46 | 129.2 | 108.1 | 111.73 | |
| 2 | 78.48 | 71.5 | 79.7 | 100.78 | 113.57 | 132.27 | |
| 3 | 68.55 | 86.94 | 79.25 | 101.16 | 103.12 | 143.59 | |
| 4 | 80.14 | 90.9 | 79.11 | 96.75 | 118.71 | 117.84 | |
| 5 | 77.06 | 78.14 | 100.08 | 116.68 | 122.58 | 122.98 | |
| 6 | 81.94 | 74.54 | 107.83 | 122.04 | 125.33 | 134.3 | |
| 7 | 72.28 | 72.17 | 84.06 | 129.2 | 129.08 | 117.19 | |
| 8 | 77.78 | 67.87 | 76.7 | 112.35 | 115.51 | 112.5 | |
| 9 | 69.77 | 92.52 | 83.66 | 103.94 | 116.89 | 112.32 | |
| 10 | 87.58 | 71.78 | 82.41 | 126.36 | 118.71 | 112.04 | |
| | 77.248 | 77.917 | 85.626 | 113.846 | 117.160 | 121.676 | Av. |
| | 5.754 | 8.904 | 10.117 | 12.594 | 7.731 | 11.313 | S.D. |

| | | | | |
|---|---|---|---|---|
| Av. S.D. | 80.264 | μm | 117.561 | μm |
| | 8.258 | | 10.546 | |

(S.D.: Standard Deviation)

### Example 17

### Putrescine and L-Ascorbic Acid Improve Skin Appearance In Vivo

### Patients and Methods

Patients were invited to participate in a topical skin preparation study in a single center. The three individuals that met the criteria were chosen at random, consisting of one male and two females, ages 33, 46 & 66, all Caucasian. Exclusion criteria included skin disease, allergies or sensitivities to topical skin preparations, recent excessive sun exposure, pregnancy, nursing, using topical prescription treatments, or any significant systemic illness or conditions. The study does not take into account daily exposure of the treated area to the environment, repeated skin cleansing or makeup application, however patients were asked to avoid excessive sun or environmental exposure and disclose any inadvertent skin irritation or injury. At baseline, a dermatological examination was performed and a brief medical history recorded. Participants were instructed to avoid any other topical skin products beyond a cleanser and apply the product for study (the formulation of Example 6) widely on the lateral canthus areas and dorsum of both hands twice daily. The areas were evaluated on a 10-point scale for texture, tone, thickness, dryness, oiliness, aging progression and healing ability: 1 being very poor and 10 being very good. Both the patients and investigator completed separate evaluation sheets and a high-resolution photographic image was taken of the left canthus and the left hand, at intervals of baseline, 4 weeks 8 weeks and 12 weeks. The images were scrambled and a blinded investigator also completed an evaluation of each image. Tolerability and safety assessments were based on patient and investigator evaluation at 4, 8 and 12-week intervals of the absence of skin irritation and the absence of adverse events. Adherence to therapy was assessed through regular follow up. Results are reported in FIGS. 4A-I.

### Results

Full compliance was achieved from all patients during the course of the study and no adverse events or experiences were reported. Based on patient, investigator and blinded evaluation, there was generally improvement of each parameter: texture, tone, thickness, dryness, oiliness and aging progression over the course of the study. Patient and investigator evaluations were most indicative of improvement, while blinded evaluations were more modest. All patients perceived an overall skin improvement, with improved skin thickness, smoothness and moisture content (See FIGS. 3A-F and 4A-I). In conclusion, to patients and the investigators in particular, the skin's age appeared to have returned to a more youthful state.

Independently of the above clinical assay, a fourth Caucasian female subject (age 45 began applying the putrescine and L-ascorbic acid serum once daily in the morning on the right side of her face and on the back of her left hand as a personal experiment (leaving the right side untreated for the hand and left side for the face). After 8 weeks of treatment, the subject began noticing improvements on the overall appearance of her skin. Noticeable improvements included skin tone, and wrinkle reduction. The subject reported continued improvements of the look of her treated skin over a period of 16 weeks and indicated that surprisingly, co-workers and friends could identify the side of her face and the hand that was treated with the serum formulation.

### CONCLUSIONS

The use of Polyamine-DAB™ (1,4-Diaminobutane) combined with L-Ascorbic Acid USP was shown to be safe, with clear indications of skin improvement during the 12-week course of use in patients. Blinded investigator evaluation was limited to photography alone, while many of the parameters of skin improvement can be best assessed by personal and tactile evaluation. Accordingly, results from blinded evaluation may underestimate the actual skin improvements following serum treatment. Each patient indicated satisfaction with product use and expressed confidence in positive skin changes during the course of the study. The scope of the claims should not be limited by the preferred embodiments set forth in the examples, but should be given the broadest interpretation consistent with the description as a whole.

### REFERENCES

1. Tajima S, Pinnell S R, Ascorbic acid preferentially enhances type I and III collagen transcription in human skin fibroblasts. J. Derm Science. 11:250-53, 1996. 2Traikovich SS.
2. Use of topical ascorbic acid and its effects on photo damaged skin topography. Arch Otolaryngol Head Neck Surg 125:1091-98, 1999.
3. Murray J, Darr D, Reich J. Pinnell S. Topical vitamin C treatment reduces ultraviolet B radiation-included erythema in human skin. J. Invest Dermatol 1991:96:587 (abstract).
4. C. W. Lynde. Moisturizers: What they are and how they work. Skin Therapy Letter, 2001; http://www.skintherapyletter.com/2001/6.13/2.html

1. A slow release aqueous topical composition comprising (i) a primary polyamine; (ii) Vitamin C; (iii) ethoxydiglycol; and (iv) a glycol selected from (a) propylene glycol, (b) butylene glycol, (c) pentylene glycol, and (d) a combination of at least two of (a) to (d).

2. A slow-release aqueous topical composition comprising (i) at least 16% w/w of vitamin C; (ii) ethoxydiglycol; and (iii) a glycol selected from (a) propylene glycol, (b) butylene glycol, (c) pentylene glycol, and (d) a combination of at least two of (a) to (d).

3. A slow-release aqueous topical composition comprising (i) at least one active ingredient, (ii) ethoxydiglycol; (iii) a glycol selected from (a) propylene glycol, (b) butylene glycol, (c) pentylene glycol and (d) a combination of at least two of (a) to (d); and (iv) betaine and/or polysorbate 20.

4. The topical composition of claim 3, wherein the composition further comprises between about 0.5 w/w and about 3% w/w of betaine; or about 1.2% of betaine.

5. (canceled)

6. The topical composition of claim 3, further comprising between about 0.1% w/w and about 3% w/w of polysorbate 20; or about 1.3% w/w of polysorbate 20.

7. (canceled)

8. The topical composition of claim 3, wherein (i) the at least one active ingredient comprises (a) a primary polyamine; or wherein said primary polyamine is a primary aliphatic lower-alkyl (C1-5) monoamine; a primary aliphatic alkylamine or a primary aliphatic lower-alkyl (C1-5) polyamine; or wherein said primary aliphatic lower-alkyl (C1-5) monoamine is aminoacetonitrile, said primary aliphatic alkylamine is spermine or spermidine and said primary aliphatic lower-alkyl (C1-5) polyamine is putrescine or dansylcadaverine, or wherein said primary polyamine putrescine (1,4-diaminobutane); and/or (b) a Vitamin C, and preferably L-ascorbic acid, 3-O-ethyl ascorbic acid (ETVC) or a combination thereof.

9. (canceled)

10. (canceled)

11. (canceled)

12. The topical composition of claim 8, wherein the at least one active ingredient comprises between about 0.1% w/w to about 1% w/w of putrescine; about 0.4% w/w of putrescine; or about 0.8% w/w of putrescine.

13. (canceled)

14. (canceled)

15. (canceled)

16. The topical composition of claim 1, wherein said composition comprises more than zero and up to about 20% w/w of 3-O-ethyl ascorbic acid (ETVC), L-ascorbic acid or a combination thereof as Vitamin C; or about 10% w/w or about 20% w/w of ETVC, L-ascorbic acid or a combination thereof as Vitamin C; or about 5% or 10% w/w of L-ascorbic acid or of ETVC; or about 5% w/w of L-ascorbic acid and 5% w/w of ETVC; or about 12.5% w/w of L-ascorbic acid and 7.5% w/w of ETVC; or about 3% w/w of L-ascorbic acid and 7% w/w ETVC.

17. (canceled)

18. (canceled)

19. (canceled)

20. (canceled)

21. (canceled)

22. (canceled)

23. (canceled)

24. (canceled)

25. The topical composition of claim 1, wherein (a) the ethoxydiglycol concentration in the composition is of between about 22% w/w and about 60% w/w; or between about 48% w/w and about 55% w/w; or about 49% w/w; or about 43% w/w; or about 33% w/w; or about 51% w/w; or more than zero and up to 2.5%; or about 2.5%; and/or (b) the glycol comprises propylene glycol in a concentration in the composition of between about 20% w/w and about 30% w/w of propylene glycol; or between about 26% w/w and about 27.5% w/w of propylene glycol; or between about 23% w/w and about 27.5% w/w of propylene glycol; or about 27%, 26% or 23% w/w of propylene glycol.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The topical composition of claim 25, wherein the glycol consists of a combination of (a) propylene glycol, (b) butylene glycol, and (c) pentylene glycol.

40. The topical composition of claim 39, wherein (i) the concentration of propylene glycol in the composition is about 32% w/w; (ii) the concentration of butylene glycol in the composition is about 22% w/w.

41. (canceled)

42. The topical composition of claim 39, wherein the concentration of pentylene glycol in the composition is about 22% w/w.

43. The topical composition of claim 39, wherein the ratio of propylene glycol:butylene glycol:pentylene glycol is about 1.45:1:1 w/w.

44. The topical composition of claim 1, wherein the composition further comprises at least one antioxidant.

45. The topical composition of claim 44, wherein at least one antioxidant comprises a fruit extract, hydroquinone, vitamin E, a retinoid or any combination thereof.

46. The topical composition of claim 45, wherein at least one antioxidant comprises an antioxidant fruit extract.

47. The topical composition of claim 46, wherein the antioxidant fruit extract is a grapefruit extract.

48. The topical composition of claim 47, comprising about 1% of grapefruit extract.

49. The topical composition of claim 1, wherein the pH of the composition is between about 2 and about 4.

50. (canceled)

51. (canceled)

52. (canceled)

53. (canceled)

54. (canceled)

55. Method if using the topical composition as defined claim 1, for (A) treating or preventing skin inflammation, skin irritation, and/or skin's signs of aging; (B) promoting wound healing; (C) preventing or reducing the formation of hypertrophic scar tissue; or (D) (i) increasing the thickness of the skin; (ii) increasing skin tone or preventing loosening of the skin; (iii) reducing dryness of the skin; (iv) reducing oiliness of the skin; and/or (v) preventing or reducing the appearance of fine lines and wrinkles.

56. (canceled)

57. (canceled)

58. (canceled)

59. (canceled)
